SEQUENCE LISTING (2 sequences)

SEQ ID NO: 1 - protein, 1704 aa, Aspergillus nidulans
MASPIIQPAGAGIHDIFTQLELWESIDKGLSMITILRDNDVLWKPFLQLTLFNQLNIVRKAWSATIQKASESDKVPTLKDVYTSESSFIAQALLDTKNLQITPPATPRTALSGALLAKTIVIFHHSERAQEELGTELPEEVRSLVNQNAICLKVLYNANQWHIDLHYKRDSLSSAQAGEVAEIFEQYLEEALEAVASAIPPSPPVEDDNAGHGGLCKERTDCPKVNRCIHDLIEEQAIARPDQEGICAYDGSLSYAGLSKLSSVLAEQLKTFGARPEQRVAILMNKSFWYPVVVLAVLKSGAAFVPLDPSHPKNRLKQLISEIEPCALITTSVLSELADDLGCPSLAIDSDLTRSKEGSTTALLPNTSASPNNAAYIIFTSGSTGKPKGVVVEHSALSTSAITRGVVLGLGPDSRVLQYAPHTFDVSVDEILTTLIHGGCVCVPSEDDRFSIAHFMESARVTVALLTPTSARTLHPDEVPSLRILQTGGEVLTEDVNDKWSNRVTLFNVYGPTEASVACVISNRTGLKGAGHVLGQAVGGKLWIVDPDDIERHLPDNEVGELVISGAILARGYFRDPSRTESSFVRMRNGERVYRTGDLASMDSAGTIIYHGRKDLEVKIRGQRINIAEIEIAILQCDLVHSVVVEYPRSGLFEKKLVAVLRFEDSSSDAEDGLFGGAKGLTEDIYCLLLSHVSSVLTPAMIPSKWLSLPCVPQMPSGKADRKQVRGWLEDMDKRTYTRIFHPNGTDNLISDPSDSMVAIWLKVLKLEPQSLRLDQSFIRNGGDSIMAMEARHQAHEAGINIDVRELLGSRALQEIGEMATKTSAVEEVSKIEDDRDEPFPLSPVQQMYFDKVSDPSLGLQQRVCVEIMTKIQPDMLREALNHVIQKHRMLAARFTKHMGQWMQQVPFGKNLKHLSRCHIYSQAVGSLGDFCSEPMALEDGTLLHAHLQSSGERQTLVLCVHHLVVDFVSWRVILQDLHDALAAAQNGLPSGISRSTLTFQQWCREQTKYASTLIPEAVLPFAPGPVNLRFWQPSNVQAVSNTYSEIVQHDFRLSSTQTTQMLEKFTTATVHPTDLMLATFALAFKRIFTERDTPTIFIEGHGREPWHASLDVSQTVGWFTAAFPIHLPKDTLLNTTTAILGASERRRSVLANGHPYWACRYLSPNGQKVFGDDPRHQEMEFVFNYAGSIVQRAPGQTLFAENVRIAEIGHPNCERFSLFDIGAAIEMPSSELVVSFTFPKGIAHRERVAELVKTYQELLETAVERDLDLSAKLSSPLVCPADVVRSLEVNGVCIERDVEIVYTPSSIQQHMLWRQSQEPWFYRVQGDWTIEKTTTQSEPVDIDRLSHAWNQVVHRHTTLRTVFRYSSEEERFVAIVLHEVKPAISIIRKGIQTSGSLCRDDDLSPPHRMVLREKDNGSVVCELEFSHTIIDAASRSIVVQDLLDAYDGKLAHRPLDFPPFWEYIRLAQSSTPSARKEELHRAGRVVTLPFQPTHVLSKVPEACKKNEITISSFFMTAWSIVLAKHFVAHNQRVDSTSSQAVAFDYVLSDRSANIPGIESAVGPYIRLPTLETHVKEGVSLKNIARGLHAQCTFQSLSQSTQDGSSLELPSKATALQKYSTLVNIRNSGSDSLDLVSDSGEWKWILQGFSDPWDYDLVFAVNVHAGKVTGWTVEYADGVVEHSAADEIAKDLNDVVERMVCEII

SEQ ID NO: 2 - protein, 1715 aa, Artificial Sequence
  Description of Artificial Sequence Synthetic polypeptide
MASHHHHHHHHTASPIIQPAGAGIHDIFTQLELWESIDKGLSMITILRDNDVLWKPFLQLTLFNQLNIVRKAWSATIQKASESDKVPTLKDVYTSESSFIAQALLDTKNLQITPPATPRTALSGALLAKTIVIFHHSERAQEELGTELPEEVRSLVNQNAICLKVLYNANQWHIDLHYKRDSLSSAQAGEVAEIFEQYLEEALEAVASAIPPSPPVEDDNAGHGGLCKERTDCPKVNRCIHDLIEEQAIARPDQEGICAYDGSLSYAGLSKLSSVLAEQLKTFGARPEQRVAILMNKSFWYPVVVLAVLKSGAAFVPLDPSHPKNRLKQLISEIEPCALITTSVLSELADDLGCPSLAIDSDLTRSKEGSTTALLPNTSASPNNAAYIIFTSGSTGKPKGVVVEHSALSTSAITRGVVLGLGPDSRVLQYAPHTFDVSVDEILTTLIHGGCVCVPSEDDRFSIAHFMESARVTVALLTPTSARTLHPDEVPSLRILQTGGEVLTEDVNDKWSNRVTLFNVYGPTEASVACVISNRTGLKGAGHVLGQAVGGKLWIVDPDDIERHLPDNEVGELVISGAILARGYFRDPSRTESSFVRMRNGERVYRTGDLASMDSAGTIIYHGRKDLEVKIRGQRINIAEIEIAILQCDLVHSVVVEYPRSGLFEKKLVAVLRFEDSSSDAEDGLFGGAKGLTEDIYCLLLSHVSSVLTPAMIPSKWLSLPCVPQMPSGKADRKQVRGWLEDMDKRTYTRIFHPNGTDNLISDPSDSMVAIWLKVLKLEPQSLRLDQSFIRNGGDSIMAMEARHQAHEAGINIDVRELLGSRALQEIGEMATKTSAVEEVSKIEDDRDEPFPLSPVQQMYFDKVSDPSLGLQQRVCVEIMTKIQPDMLREALNHVIQKHRMLAARFTKHMGQWMQQVPFGKNLKHLSRCHIYSQAVGSLGDFCSEPMALEDGTLLHAHLQSSGERQTLVLCVHHLVVDFVSWRVILQDLHDALAAAQNGLPSGISRSTLTFQQWCREQTKYASTLIPEAVLPFAPGPVNLRFWQPSNVQAVSNTYSEIVQHDFRLSSTQTTQMLEKFTTATVHPTDLMLATFALAFKRIFTERDTPTIFIEGHGREPWHASLDVSQTVGWFTAAFPIHLPKDTLLNTTTAILGASERRRSVLANGHPYWACRYLSPNGQKVFGDDPRHQEMEFVFNYAGSIVQRAPGQTLFAENVRIAEIGHPNCERFSLFDIGAAIEMPSSELVVSFTFPKGIAHRERVAELVKTYQELLETAVERDLDLSAKLSSPLVCPADVVRSLEVNGVCIERDVEIVYTPSSIQQHMLWRQSQEPWFYRVQGDWTIEKTTTQSEPVDIDRLSHAWNQVVHRHTTLRTVFRYSSEEERFVAIVLHEVKPAISIIRKGIQTSGSLCRDDDLSPPHRMVLREKDNGSVVCELEFSHTIIDAASRSIVVQDLLDAYDGKLAHRPLDFPPFWEYIRLAQSSTPSARKEELHRAGRVVTLPFQPTHVLSKVPEACKKNEITISSFFMTAWSIVLAKHFVAHNQRVDSTSSQAVAFDYVLSDRSANIPGIESAVGPYIRLPTLETHVKEGVSLKNIARGLHAQCTFQSLSQSTQDGSSLELPSKATALQKYSTLVNIRNSGSDSLDLVSDSGEWKWILQGFSDPWDYDLVFAVNVHAGKVTGWTVEYADGVVEHSAADEIAKDLNDVVERMVCEII

(19) United States
(12) Patent Application Publication
(10) Pub. No.: US 2022/0315967 A1
(43) Pub. Date: Oct. 6, 2022

(54) METHODS FOR PRODUCING D-TRYPTOPHAN AND SUBSTITUTED D-TRYPTOPHANS

(71) Applicant: The Regents of the University of California, Oakland, CA (US)

(72) Inventors: Yi Tang, Arcadia, CA (US); Yang Hai, Los Angeles, CA (US)

(73) Assignee: The Regents of the University of California, Oakland, CA (US)

(21) Appl. No.: 17/640,152

(22) PCT Filed: Sep. 18, 2020
(86) PCT No.: PCT/US20/51418

§ 371 Date: Mar. 3, 2022

Related U.S. Application Data
(60) Provisional application No. 62/902,527, filed on Sep. 19, 2019.

(51) Int. Cl. C12P 13/22; C12N 9/00; C12N 9/10

(52) U.S. Cl. CPC C12P 13/227; C12N 9/93; C12N 9/1029; C12Y 203/01048; C12R 2001/865

(57) ABSTRACT

Single-module nonribosomal peptide synthetases (NRPSs) and NRPS-like enzymes activate and transform carboxylic acids in both primary and secondary metabolism; and are of great interest due to their biocatalytic potentials. The single-module NRPS IvoA is essential for fungal pigment biosynthesis. As disclosed herein, we show that IvoA catalyzes ATP-dependent unidirectional stereoinversion of L-tryptophan to D-tryptophan with complete conversion. While the stereoinversion is catalyzed by the epimerization (E) domain, the terminal condensation (C) domain stereoselectively hydrolyzes D-tryptophanyl-S-phosphopantetheine thioester and thus represents a noncanonical C domain function. Using IvoA, we demonstrate a biocatalytic stereoinversion/deracemization route to access a variety of substituted D-tryptophan analogs in high enantiomeric excess.

### CROSS REFERENCE TO RELATED APPLICATIONS

This application claims the benefit under 35 U.S.C. Section 119(e) of co-pending and commonly-assigned U.S. Provisional Patent Application Ser. No. 62/902,527 filed on Sep. 19, 2019 and entitled “METHODS FOR PRODUCING D-TRYPTOPHAN AND SUBSTITUTED D-TRYPTOPHANS” which application is incorporated by reference herein.

### STATEMENT REGARDING FEDERALLY SPONSORED RESEARCH AND DEVELOPMENT

This invention was made with government support under Grant Number GM 118056, awarded by the National Institutes of Health. The government has certain rights in the invention.

### TECHNICAL FIELD

The present invention relates to methods and materials useful for making D-tryptophans and substituted D-tryptophans.

### BACKGROUND OF THE INVENTION

D-tryptophans are important building blocks of many peptide-based pharmaceuticals such as tadalafil (FDA approved for the treatment of erectile dysfunction and benign prostatic hyperplasia), macimorelin (FDA approved for the diagnosis of adult growth hormone deficiency), triptorelin (FDA approved for the treatment of advanced prostate cancer), parsireotide (FDA approved orphan drug for the treatment of Cushing's disease in patients who are ineligible for surgical therapies), lanreotide (FDA approved for the treatment of acromegaly), and octreotide (FDA approved for the treatment of acromegaly and diarrhea associated with certain types of tumors) (see FIG. 5).

To date, a number of biocatalytic processes for the synthesis of D-tryptophan have been developed. For example, Yamamoto et al. described the use of bacterial tryptophanase to selectively degrade L-tryptophan from a racemic mixture of D,L-tryptophan resulting in enrichment of D-tryptophan (U.S. Pat. No. 5,916,781): Mitsuhashi et al. described the use of fungal aminoacylase to selectively deacetylate N-acetyl-D-tryptophan thus to enrich D-tryptophan from a racemic mixture of N-acetyl-D,L-tryptophan (U.S. Pat. No. 6,780,619). These methods are all based on kinetic resolution by using an enantioselective enzyme. One common disadvantage exists with such conventional methodologies, in that the theoretical yield of D-tryptophans is always limited to 50%. To overcome this intrinsic shortcoming, dynamic kinetic resolution and stereoinversion reaction cascades have been developed. For example, Parmeggiani et al. reported the combination of L-amino acid deaminase and engineered D-alanine amino transferase to realize stereoinversion and deracemization in making substituted D-tryptophans (Parmeggiani et al., Chem Rev. 2018 Jan. 10; 118(1):73-118). However, not one of these conventional methods is able to convert L-tryptophan to D-tryptophan in a one-step process. One step processes in such chemosynthetic technologies are highly desirable in that they provide artisans with cost effective and green alternatives to multistep processes which necessarily require additional resources, and generate more waste.

For the reasons noted above, there is a need in the art for methods and materials useful for converting L-tryptophan into D-tryptophan as well as substituted L-tryptophans into substituted D-tryptophans in a one-step process.

### SUMMARY OF THE INVENTION

As discussed in detail below, we have developed a system that uses a single-module nonribosomal peptide synthetase IvoA derived from Aspergillus nidulans to produce D-tryptophan and substituted D-tryptophans. The invention disclosed herein offers a concise one-step, direct nonredox stereoinversion/deracemization process for generating D-tryptophans, and further allows artisans to generate libraries of D-tryptophan analogues in high enantiomeric excess at millimolar levels. In this way, the invention disclosed herein provides a new and unique platform for the cost-effective synthesis of a variety of peptide-based molecules.

The invention disclosed herein has a number of embodiments. Embodiments of the invention include, for example, methods of making D-tryptophan or substituted D-tryptophan analogs by combining L-tryptophan or a substituted L-tryptophan analog with a single-module nonribosomal peptide synthase IvoA polypeptide such that the IvoA polypeptide catalyzes unidirectional stereoconversion of the L-tryptophan to a D-tryptophan or the substituted L-tryptophan analog to a substituted D-tryptophan analog; so that the D-tryptophan or the substituted D-tryptophan analog is made. In an illustrative working embodiments of the invention, single-module nonribosomal peptide synthase ivoA polypeptides (“IvoA”) were expressed in recombinant yeast cells in the presence of L-tryptophan; and free D-tryptophan was then isolated from these yeast cells at yield of 5-10 mg/L.

In certain embodiments of the invention, the single-module nonribosomal peptide synthetase ivoA polypeptide comprises an amino acid sequence having at least a 90% identity to wild type Aspergillus nidulans 1704 amino acid wild type IvoA polypeptide (SEQ ID NO:1). In typical embodiments of the invention, at least 90% of the L-tryptophan or a substituted L-tryptophan is converted to D-tryptophan or a substituted D-tryptophan. In certain embodiments of the invention, the method is performed on a substituted D-tryptophan analog such as a 5-OMe-L-tryptophan, a 4-F-L-tryptophan, a 5-F-L-tryptophan, a 6-F-L-tryptophan, a 5-Cl-L-tryptophan, a 6-Cl-L-tryptophan, a 5-Br-L-tryptophan, a 4-Me-L-tryptophan, a 5-Me-L-tryptophan, a 6-Me-L-tryptophan, or a 7-Me-L-tryptophan. In certain embodiments of the invention, a D-tryptophan is made via fermentation in a yeast strain selected to overexpress an IvoA polypeptide. In working embodiments of the invention disclosed herein, the yeast strain comprises an Aspergillus nidulans phosphopantetheinyl transferase gene; a mutated histone acetyltransferase hpa3 gene; and a heterologous leu2 gene. In this working embodiment, the yeast strain produced at least 5 mg/L of D-tryptophan in culture. In other embodiments of the invention, one or more substituted D-tryptophans can similarly be made via fermentation by feeding the one or more L-tryptophan analogues into a yeast strain culture, wherein the yeast strain is selected to overexpress an IvoA polypeptide.

Another embodiment of the invention is a system or kit for generating a D-tryptophan or a substituted D-tryptophan analog comprising a first container comprising a single-module nonribosomal peptide synthase ivoA polypeptide or a polynucleotide encoding a single-module nonribosomal peptide synthase ivoA polypeptide. In certain embodiment of the invention, the system or kit comprises a yeast strain that overexpresses an IvoA polypeptide.

Without being bound by a specific theory or mechanism of action, a proposed mechanism of IvoA catalyzed stereoinversion is summarized in FIG. 6B. Briefly, the adenylation (A) domain activates L-tryptophan via adenylation reaction and tethers the activated L-tryptophan to the phosphopantetheine (Ppant) arm of the thiolation (T) domain in the thioester linkage. The T domain channels the reactive thioester to epimerization domain (E) and the L-tryptophanyl-S-Ppant thioester gets epimerized to yield a mixture of D, L-tryptophanyl-S-Ppant in equilibrium. Finally, the releasing condensation (C) domain stereoselectively hydrolyzes the D-tryptophanyl-S-Ppant to complete the stereoinversion. Note that D-tryptophan is activated by IvoA with lower efficiency. The loaded D-tryptophan can approach the same internal equilibrium of L/D-tryptophanyl-S-Ppant thioesters via epimerization. However, the D-specific C domain ensures only D-tryptophanyl-S-Ppant thioester is hydrolyzed. The prominent activity of IvoA is further demonstrated by the ability to deracemize a library of substituted tryptophans in affording the corresponding D-enantiomers in high enantiomeric excess (Table 2).

In illustrative working embodiments of the invention discussed below, we genetically modified a yeast strain to overexpress IvoA in order produce D-tryptophan directly from yeast fermentation. In this embodiment, the A. nidulans phosphopantetheinyl transferase gene npgA was integrated to the yeast genome and the histone acetyltransferase hpa3 in the yeast genome was replaced with leu2. The resulting strain was transformed with plasmid carrying ivoA gene regulated under a ADH2 promoter. The recombinant strain was cultured in YPD medium for 3 days. Free D-tryptophan was then isolated from the yeast cells at yield of 10 mg/L. Purified D-tryptophan was shown to be in high enantiomeric excess (ee=98%) by chiral-HPLC analysis.

Embodiments of the invention disclosed herein have a number of applications. For example, embodiments of the invention can use IvoA protein as a biocatalyst for synthesis of an array of D-tryptophans through ATP-dependent direct stereoinversion or deracemization. In view of this discovery, D-tryptophan and substituted D-tryptophans having substituents of either electron-withdrawing or donating groups, at most positions of the indole ring (e.g. positions 4, 5, 6 and 7) can now be accessed in high enantiomeric excess (>99%). In addition, embodiments of the invention are readily scalable and, for example, can use genetically modified yeast strains selected to overexpress IvoA to generate significant amounts of D-tryptophan or substituted D-tryptophan analogs by fermentation. Embodiments of the invention can use IvoA protein as a biocatalyst for synthesis of α-deuterium-labeled D-tryptophans and α-deuterium-labeled substituted D-tryptophan analogs by performing the reactions in deuterium oxide instead of water, as demonstrated in FIG. 2B and FIG. 2C.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIGS. 1A and 1B provide schematics showing the diverse functions of single-module NRPS and NRPS-like enzymes. FIG. 1A provides characterized examples and FIG. 1B shows IvoA studied in this work.

FIGS. 2A-2D provide data showing the characterization of IvoA activity. FIG. 2A provides the Stereochemistry determination for isolated N-acetyl-D-tryptophan. FIG. 2B provides the Mass spectrometry showing the mass shift of tryptophan when the assay was performed in D₂O. FIG. 2C provides the ¹H-NMR spectra indicate incorporation of deuterium at the α position: 1) the change of splitting pattern of the diastereotopic β proton signal due to smaller coupling constant (³J_{H-D})); 2) the disappearance of a proton signal. FIG. 2D provides the Chiral HPLC resolution of tryptophan enantiomers from IvoA reaction demonstrated complete stereoinversion of L-tryptophan to D-tryptophan.

FIG. 3 provides a schematic showing a working model of IvoA.

FIG. 4 provides schematics and data showing a Characterization of IvoA-C activity in vitro by LC-MS. D-Trp-S-Ppant conversion to D-tryptophan is shown in the left panel, and L-Trp-S-Ppant conversion to L-tryptophan is shown in the right panel.

FIG. 5 provides a schematic showing FDA approved drugs containing D-tryptophan building blocks. D-tryptophans are important building blocks of many peptide-based pharmaceuticals such as those shown in this Figure.

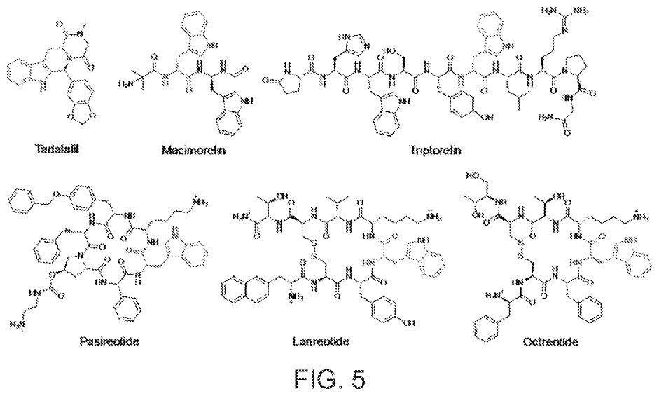

FIGS. 6A and 6B provide data and a schematic showing IvoA catalyzed ATP-dependent stereoinversion of L-tryptophan. FIG. 6A provides a Time-course reaction monitored by chiral-HPLC showing complete conversion. FIG. 6B provides a Proposed mechanism of IvoA.

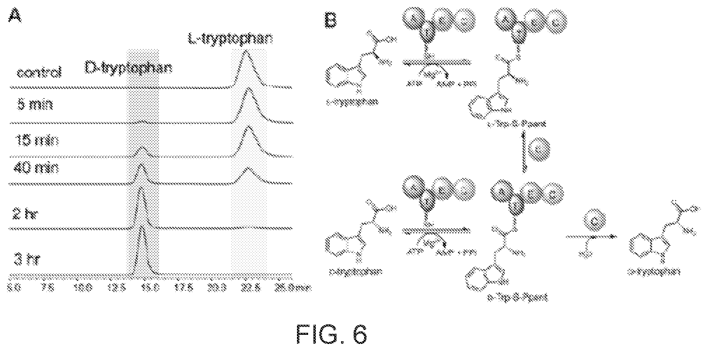

FIG. 7 provides a Schematic of embodiment of the invention.

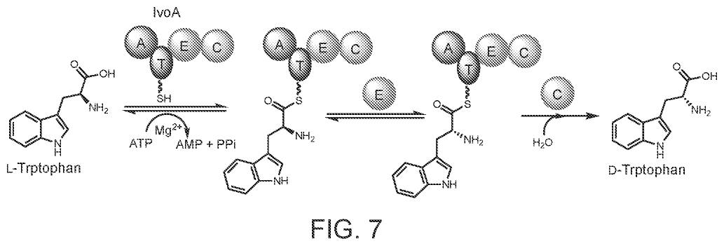

FIG. 8 provides data showing SDS-PAGE analysis of purified IvoA proteins. NuPAGE™ 4-12% Bis-Tris protein gels were used for analysis and gels were running with NuPAGE™ MES SDS running buffer.

FIG. 9 provides data showing Hydroxylamine-based colorimetric assay for studying substrate specificity of IvoA A domain. The reaction condition is 2 μM of IvoA-T⁰+3 mM of ATP+0.1 mM of carboxylic acid substrate+15 mM hydroxylamine+15 mM MgCl₂ in Tris buffer (pH 8.0). The reaction is quenched after 3 hrs by addition of equivalent volume (150 μL) of stopping solution (10% (w/v) FeCl₃.H₂O and 3.3% (w/v) trichloroacetic acid dissolved in 0.7 M HCl). The precipitated enzyme was removed by centrifugation and the supernatant was measured for its absorbance at 540 nm.

FIG. 10 provides data showing In vitro characterization of IvoA acetyltransferase activity by HPLC. The reaction condition is 100 μM of IvoA+2 mM of ATP+1 mM of L-tryptophan+1 mM of AcCoA+5 mM MgCl₂ in phosphate buffer (pH 7.5). Each trace represents: i) N-acetyltryptophan standard; ii) 30 min reaction; iii) 24 hrs reaction; iv) 24 hrs reaction using boiled enzyme.

FIG. 11 provides data showing the Apparent steady-state kinetics of IvoA catalyzed stereoinversion. Substrate inhibition was observed with WT enzyme. The kinetic constants were shown in the main text. For WT, 1 μM enzyme was used in each assay and the reaction was quenched after 2 min. For E0 mutant, 10 μM enzyme was used in each assay and the reaction was quenched after 60 min. For C0 mutant, 50 μM enzyme was used in each assay and the reaction was quenched after 60 min.

FIG. 12 provides data (left panel) and an associated schematic (right panel) showing Gene-knockout of hpa3 in yeast. Replacement of hpa3 gene by LEU2 marker. Successful gene replacement will cause size change of PCR fragments. The integration was confirmed by colony PCR.

FIGS. 13A and 13B provide data showing the Characterization of IvoA activity in vivo. FIG. 13A provides a HPLC analysis of extracellular metabolites extracted from the culture medium. Excess L-tryptophan fed to the culture was converted to tryptophol (denoted by *). FIG. 13B provides a HPLC analysis of intracellular metabolites extracted from the yeast cell pellets.

FIG. 14 provides data showing the Chiral-HPLC analysis of purified tryptophan from yeast cells. Note that D-enantiomer is eluted earlier than L-enantiomer. The analysis was performed by using a Crownpak® CR(+) column (150 mm×4 mm×3.5 μm, Daicel) at room temperature. The mobile phase was aq. HClO₄ 1% (w/v) supplemented with 15% (v/v) MeOH and the flow rate was 1.0 ml/min.

FIGS. 15A and 15B provide data showing an In vitro hydrogen-deuterium exchange assay of IvoA with D-tryptophan. FIG. 15A provides a Mass-spectrometry analysis showing the +1 Da mass shift of tryptophan in D₂O. FIG. 15B provides ¹H-NMR spectra indicate hydrogen-deuterium exchange took place at the α position. The observation is similar to that with L-tryptophan, which indicates that epimerization also occurs with D-tryptophan as substrate.

FIG. 16 provides data (left panel) and an associated schematic (right panel) showing Complementation of IvoA mutant with standalone IvoA-C. Assay was performed by using 20 μM enzyme and 1 mM substrates. The impaired catalytic activity of C domain mutant or truncation variant can be complemented by adding standalone C domain in trans.

FIG. 17 provides data and associated schematics for an In vitro assay of IvoA-C with synthetic thioester substrates. Synthetic thioester substrates (DL-tryptophan-S—N-acetylcysteamine, DL-Trp-SNAC: D-tryptophan-S-pantatheine, D-Trp-pant) were incubated with standalone IvoA C domain at pH 6.9. Enzymes were boiled to measure the nonenzymatic hydrolysis. Free tryptophan and tryptophanyl thioesters were separated by HPLC. The enzyme catalyzed hydrolysis rate is not significantly different from noncatalyzed reaction, which indicates that these synthetic thioesters are not good substrate for IvoA-C.

FIG. 18 provides data and schematics showing Loading of D/L-tryptophan to IvoAΔC monitored by intact protein mass spectrometry. Intact mass spectra of holo-IvoAΔC (top), and following incubation with MgCl₂, ATP and L-/D-tryptophan (middle and bottom). Loading reaction conducted with L- and D-tryptophan resulted in a +186 Da mass shift to the intact the intact holo-IvoAΔC protein.

FIG. 19 provides data showing Adenylation assay of IvoA A domain with substituted tryptophan amino acids. The reaction was performed similarly according to the assay described in the caption of FIG. 9.

FIG. 20 provides a schematic showing Genomic context analysis of ivoA homologues found in other fungi. Numbers below open reading frame indicate the amino acid sequence identity of IvoA and IvoC homologues to A. nidulans proteins. Abbreviations to follow: ZnF, zinc-finger protein; MFS, major-facilitator superfamily; HP, hypothetical protein; IDO, indoleamine 2,3-dioxygenase; ATR, NRPS-like carboxylic acid reductase harboring domain architecture as A-T-R; Trp-DMAT, dimethylallyl tryptophan synthase-like protein; KFA, kynurenine formyl amidohydrolase.

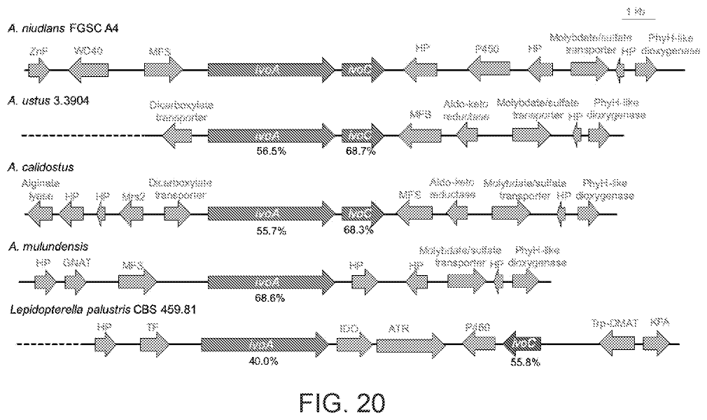

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the aspects of the techniques and procedures described or referenced herein are well understood and commonly employed by those skilled in the art. The following text discusses various embodiments of the invention.

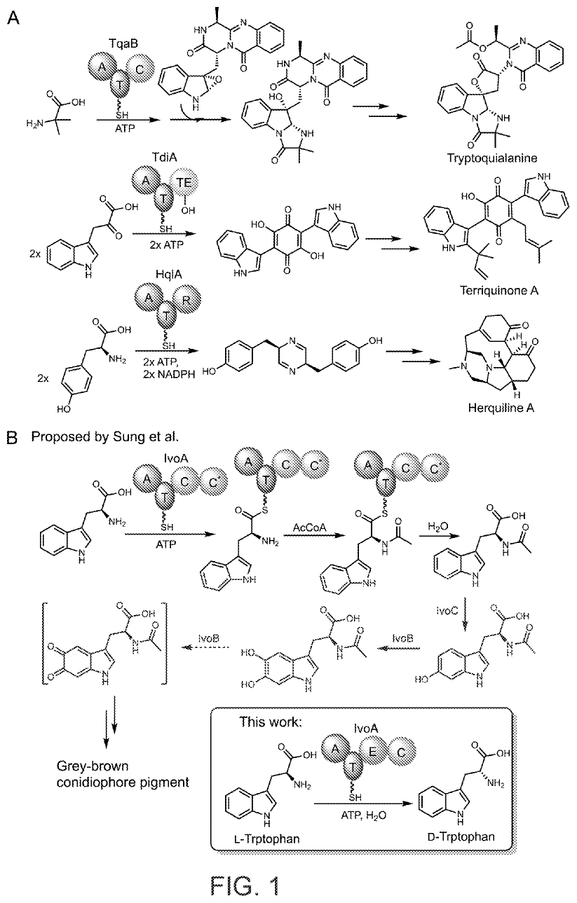

Nonribosomal peptide synthetases (NRPSs) are modular enzymes employing an assembly-line logic to synthesize a myriad of peptide-based secondary metabolites with diverse structures and biological activities (1). Single-module NRPS and NRPS-like enzymes adopt similar thiotemplated enzymology with a single set of adenylation (A) and thiolation (T) domain. These enzymes have important functions in transforming carboxylic acid substrates in primary and secondary metabolism (2); and have increased interests as biocatalysts due to their functional diversity (FIG. 1) (3) Following selection and thermodynamic activation of the carboxylic acid by the A domain, the substrate is preserved as phosphopantetheine (Ppant) thioester on the T domain. Depending on the type of downstream releasing domains, the thioester intermediates are subjected to a broad range of modifications (FIG. 1A), including but not limited to: esterification/amidation by a condensation (C) domain in A-T-C (4); Dieckman/aldol condensation or cyclization by a thioesterase (TE) domain in A-T-TE (5,6); 2- or 4-electron reduction by reductase (R) domain in either A-T-R or A-T-R-R (2b,7), 2-electron reduction followed by PLP-dependent aldol condensation in A-T-R-P (8). In natural product biosynthetic pathways, these enzymes generate natural product scaffolds with structural diversity that complements the chemical space of canonical nonribosomal peptides, such as dihydroxybenzoquinone, furanone, butyrolactone, and dihydropyrazine (FIG. 1A).

Recently, a single-module NRPS, encoded by the gene ivoA from Aspergillus nidulans with an unusual domain architecture annotated as A-T-C-C* was proposed to acetylate L-tryptophan (9). The enzymatic product N-acetyl-L-tryptophan was suggested to be further oxidized by a P450 enzyme IvoC and a phenol oxidase IvoB en route to the conidiophore pigment (FIG. 1B). Although genetic studies provided compelling evidence implicating ivoA in the biosynthesis of N-acetyl-hydroxytryptophan (10,11), the proposed acetyltransferase activity of IvoA is an unlikely fit for a single-module NRPS. Furthermore, the mechanistic proposal for IvoA is at odds with accepted logic of NRPS enzymology for the following reasons: 1) It is metabolically wasteful to activate the carboxy group of a substrate at the expense of one equivalent of ATP in order to accomplish N-acetylation by acetyl-CoA; 2). It is against the NRPS directionality rule for a downstream C domain to carry out a condensation reaction (acetylation here) with an upstream T domain as the acceptor (1, 12).

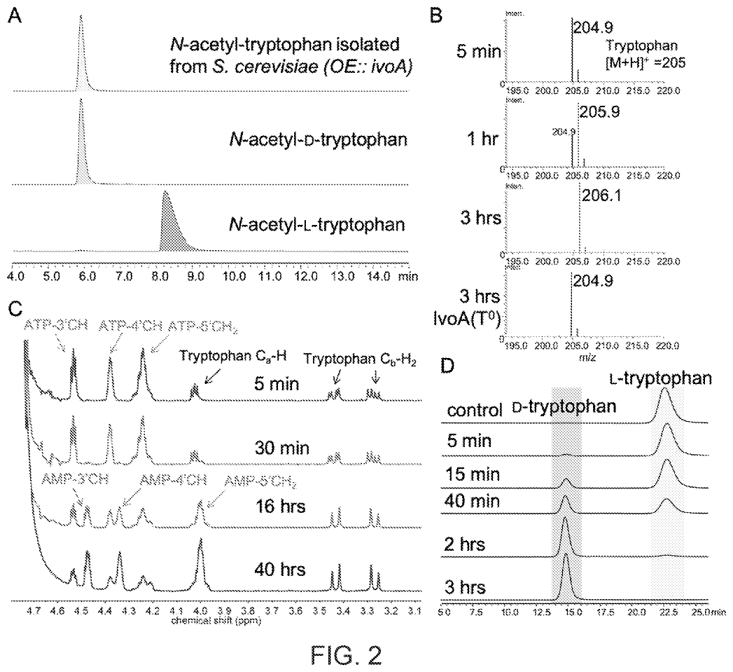

To elucidate the enzymatic function of IvoA, we first reanalyzed its domain architecture. Since epimerization (E) domains show sequence and structure homology to C domains, and are often inserted between T and C domains in the NRPS assembly lines (12), we hypothesized that the true domain organization of IvoA is A-T-E-C. Embedding a functional E domain could rationalize the necessity of involving NRPS machinery: activation of the α-carboxy group can lower the pKa of the C_{α} proton, thereby facilitating stereoinversion. To test this hypothesis, we overexpressed IvoA by using S. cerevisiae JHY686 strain as a heterologous host (13). Consistent with the previous report, we were able to detect N-acetyltryptophan formation. However, the purified product from yeast cell culture was found to be exclusively D-enantiomeric (ee>99%) as confirmed by chiral-HPLC analysis (FIG. 2A). The inverted stereochemistry of tryptophan supports our hypothesis that an E domain is present within IvoA.

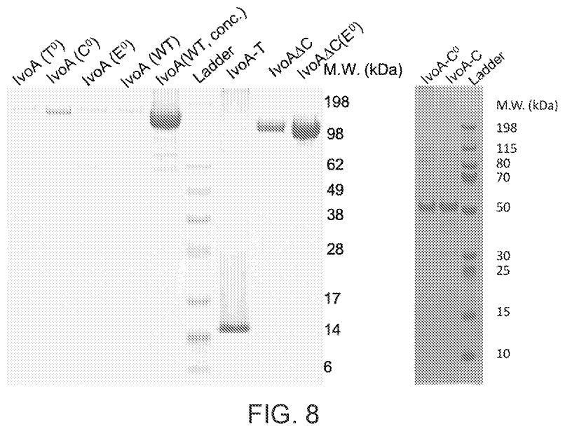

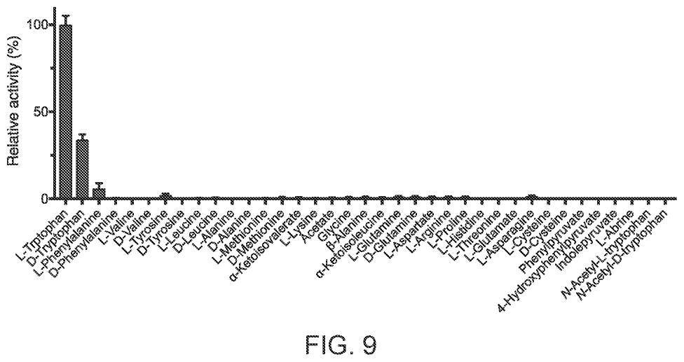

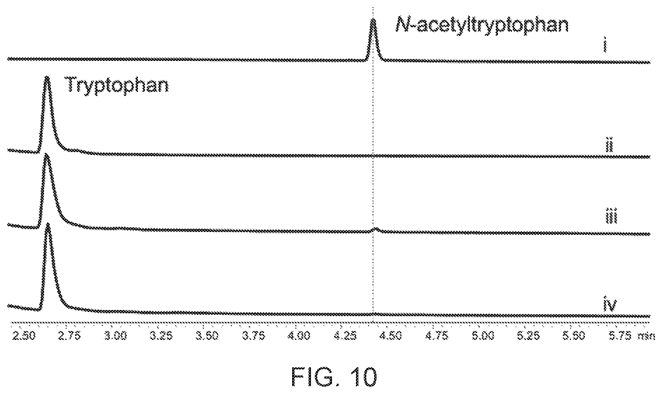

To interrogate the function of IvoA, particularly the cryptic acetyltransferase activity, we purified IvoA from S. cerevisiae and assayed its activity in vitro (FIG. 8). We first examined the substrate specificity of IvoA adenylation domain (FIG. 9). As expected, L-tryptophan is the preferred substrate, while D-tryptophan is activated with 64% lower efficiency (FIG. 9). We were unable to detect formation of acetylated tryptophan starting from either LD-tryptophan in the presence of ATP, provided with either acetyl-CoA or acetyl-phosphate as the acetyl-donor. Increasing enzyme concentration to >50 μM, plus prolonged overnight incubation only led to trace amount of N-acetyl-D-tryptophan (k_{obs}^{app}<0.1 h⁻¹, FIG. 10).

When we performed the assay ((E)=2 μM) in D₂O and analyzed the reaction mixture by LC-MS, we readily observed a gradual+1 Da mass shift of tryptophan (FIG. 2B). This deuterium “wash-in” result is phosphopantetheinylation-dependent as inactivating the T domain (IvoA-T⁰, S785A mutation) completely abolished the incorporation. Monitoring the reaction by ¹H-NMR confirmed that the hydrogen-deuterium exchange took place at the C_{α}—H (FIG. 2C), indicating that epimerization of tryptophanyl-S-Ppant occurred. The complete hydrogen-deuterium exchange is also consistent with the “two-base” mechanism proposed for NRPS E domain (14).

TABLE 1
Apparent steady-state kinetic constants of L-tryptophan
stereoinversion by IvoA and mutants.

| | k_{cat}^{app} | Kₘ^{app} | (k_{cat}/Kₘ)^{app} |
|---|---|---|---|
| Enzyme | (min⁻¹) | (μM) | (M⁻¹s⁻¹) |
| WT^{a} | 38 ± 3 | 50 ± 10 | 1.3 × 10⁴ |
| T⁰(S785A) | inactive | inactive | inactive |
| E⁰(H963A) | 0.09 ± 0.03 | 50 ± 10 | 30 |
| C⁰(H1428A) | 0.008 ± 0.002 | 40 ± 10 | 3.5 |

^{a}Substrate inhibition was observed with Kᵢ^{app} of 4 ± 1 (mM).

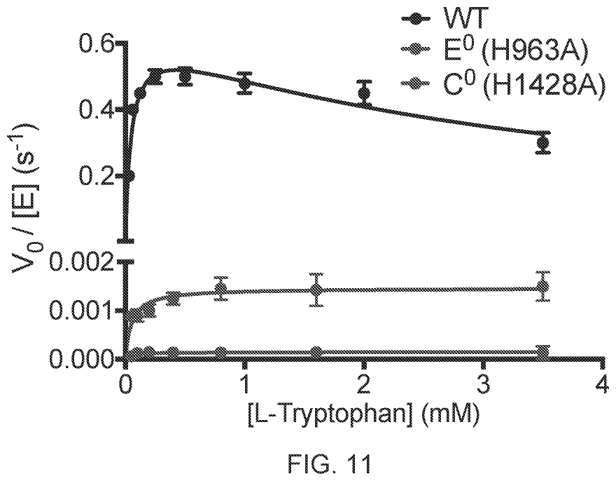

We next followed the reaction by using chiral-HPLC and complete conversion of L-tryptophan (1 mM) to D-tryptophan was observed in 3 hours (FIG. 2D). Chiral-resolution allowed us to determine the apparent steady-state kinetics by quantifying D-tryptophan formation (Table 1, FIG. 11). The apparent k_{cat} (38 min⁻¹) is similar to that of other characterized single-module NRPSs and NRPS-like enzymes (4,7), while the Kₘ (˜50 μM) is close to the intracellular level of L-tryptophan (12 μM) (15). Both E and C domains are catalytically important for IvoA, as inactivating either domain by mutating the catalytic histidine residues (H963A and H1428A) substantially compromised the apparent turnover number k_{cat} (420-fold by E⁰ while 4700-fold by C⁰). In contrast, the apparent Michaelis constants were not changed, suggesting that substrate binding (at the A domain) was not affected.

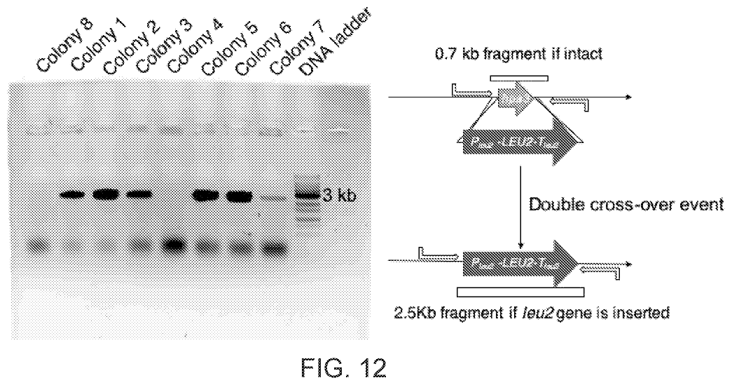

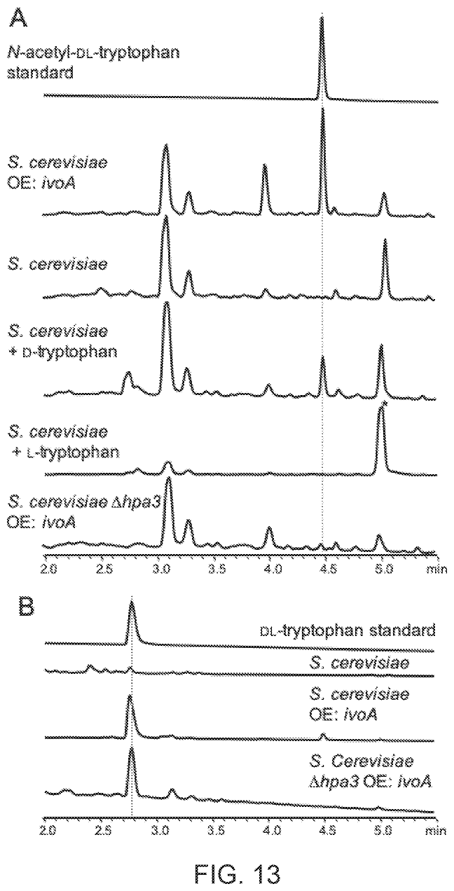

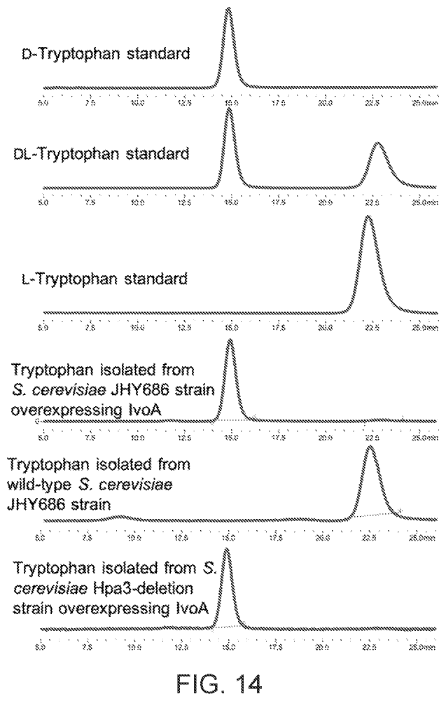

Taken together, these data indicate that IvoA lacks acetyltransferase activity in vitro, but instead is a bona fide ATP-dependent enzyme catalyzing enantioselective stereoinversion of L-tryptophan to D-tryptophan. The observed acetylation of D-tryptophan in vivo must be carried out by an endogenous acetyltransferase. Because yeast histone acetyltransferase Hpa3 is known to act as a D-amino acid N-acetyltransferase for detoxification of D-amino acids (16), we overexpressed IvoA in the hpa3-deleted yeast strain constructed by replacing hpa3 with leu2 (SI Methods, FIG. 12). Consequently, the culture medium was devoid of N-acetyltryptophan, whereas free D-tryptophan (ee=98%) were still accumulated inside the cells (FIG. 13-14). Therefore, we conclude that IvoA does not acetylate tryptophan and the origin of the negligible acetyltransferase activity of IvoA observed in vitro may derive from trace amount of contaminated yeast Hpa3.

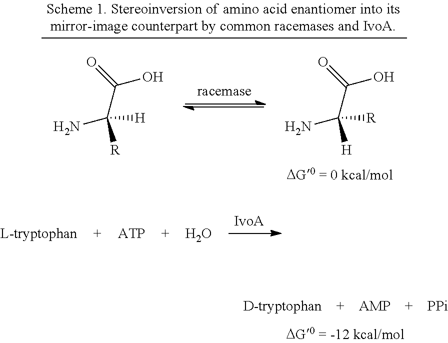

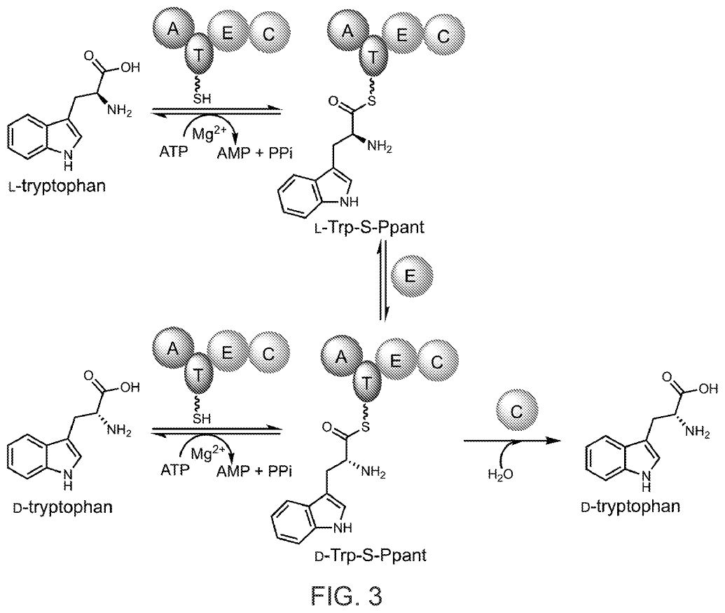

Distinct from common PLP-dependent or PLP-independent amino acid racemases (Scheme 1), which often catalyze bidirectional stereoinversion and also inevitably lead to racemization (equilibrium constant approaches unity) (17). IvoA catalyzes unidirectional stereoinversion, completely converting L-tryptophan to its enantiomer D-tryptophan. The complete conversion is driven by coupled ATP hydrolysis, which is thermodynamically favored (Scheme 1) (18), and is enabled by the thiotemplate enzymology of IvoA (FIG. 3). We reason that the activated tryptophan is delivered to the E domain as tryptophanyl-S-Ppant thioester, which undergoes epimerization to give a mixture of D/L-tryptophanyl-S-Ppant diastereoisomers in equilibrium. We propose that dynamic kinetic resolution may be accomplished by the C domain in a releasing step, which stereoselectively hydrolyzes the D-tryptophanyl-S-Ppant thioester to achieve irreversible conversion.

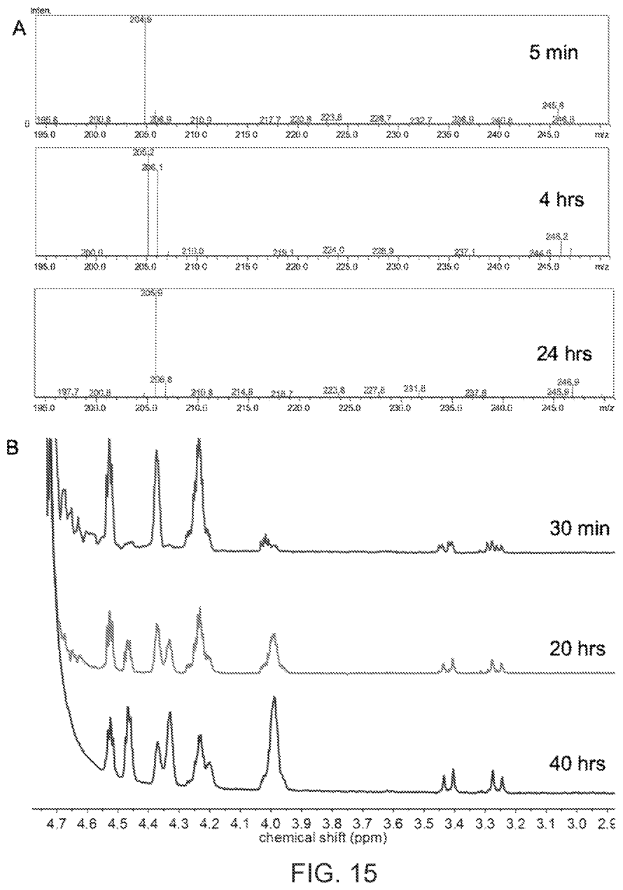

As mentioned earlier, even though IvoA A domain prefers L-tryptophan, D-tryptophan can still be adenylated and thioesterified (FIG. 9). In addition, the loaded D-tryptophanyl-S-Ppant underwent epimerization by IvoA E domain as evidenced by similar, yet slower deuterium “wash-in” behavior under multiple-turnover condition (FIG. 15). The slower turnover measured by deuterium incorporation reflects the lower adenylation efficiency of D-tryptophan. Nonetheless, the occurrence of hydrogen-deuterium exchange at D-tryptophanyl-S-Ppant Ca position not only suggests that epimerization is faster than the C-domain catalyzed D-specific tryptophanyl thioester hydrolysis, but also indicates that the D/L-tryptophanyl-S-Ppant equilibrium can be approached from either direction (FIG. 3). However, ivoA cannot convert D-tryptophan to L-tryptophan, which suggests that the L-tryptophanyl-S-Ppant is not hydrolyzed by the C domain. A D-specific hydrolytic releasing C domain is therefore the key for unidirectional complete stereoinversion.

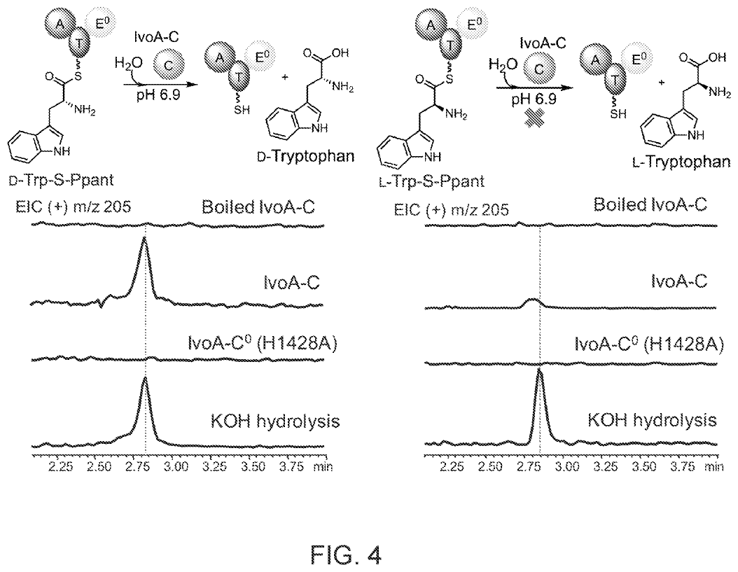

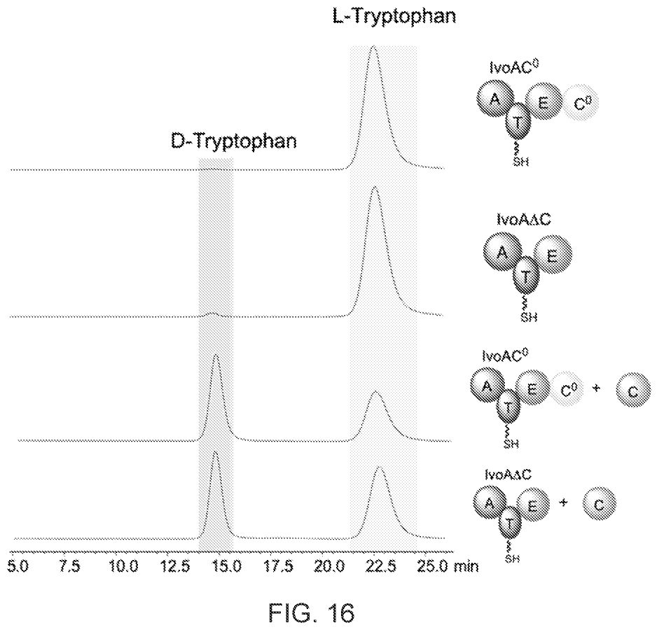

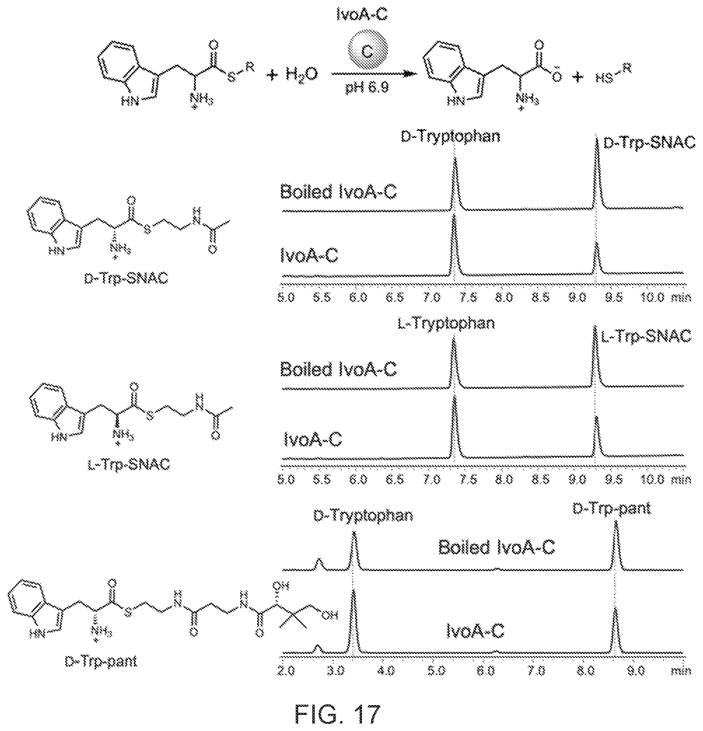

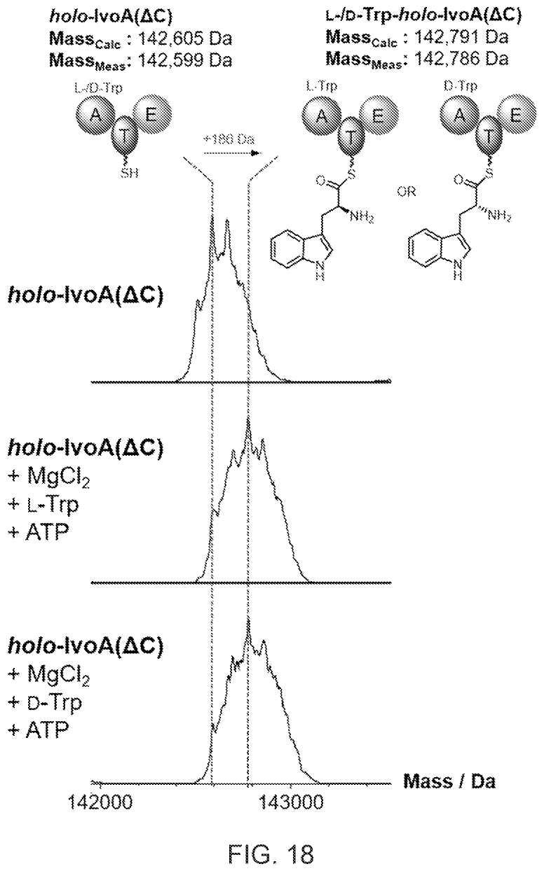

To directly demonstrate the stereoselectivity of IvoA C domain, we purified the standalone IvoA-C and assayed its activity in vitro. Addition of IvoA-C in equimolar to either IvoA(C⁰) mutant or IvoA-ΔC truncation mutant successfully rescued the impaired stereoinversion activity, which proved that the stand alone IvoA-C is active (FIG. 16). We then synthesized both D- and L-tryptophanyl-S—N-acetylcysteamine as surrogate substrates mimicking the IvoA T domain bound tryptophanyl-S-Ppant intermediates. However, the enzyme did not catalyze hydrolysis significantly above the background nonenzymatic rate (FIG. 17). Using D-tryptophanyl-S-pantetheine (D-Trp-pant) also did not improve enzymatic hydrolysis. We reason that the protein:protein interaction between T and C domain is important for substrate recognition, which has been shown in other studies of C domains (19). Hence, we chose to enzymatically load D/L-tryptophan to IvoA-ΔC(E⁰) by taking advantage of the promiscuous A domain. It is imperative to inactivate the E domain in this truncation mutant in order to minimize the epimerization. The formation of corresponding D/L-tryptophanyl-S-Ppant of IvoA-ΔC was confirmed by intact protein mass spectrometry (FIG. 18). Free excess D/L-tryptophan substrates were quickly removed from IvoA-ΔC(E⁰) by using desalting spin columns and the loaded D/L-tryptophanyl-S-IvoA-ΔC(E⁰) were immediately subjected to IvoA-C catalyzed hydrolysis. The liberated free tryptophan was then quantified by LC-MS. As shown in FIG. 4, IvoA-C stereoselectively hydrolyzed D-tryptophanyl-S-IvoA-ΔC(E⁰) over L-tryptophanyl-S-IvoA-ΔC(E⁰). NRPS C domains that have thioesterase activity are rare, and to date only one example from crocacin PKS-NRPS hybrid assembly-line was known, but did not show stereoselectivity (20). Therefore, the IvoA-C characterized here represents a novel C domain and we classify it as a ^{D}C_{H2O} subtype according to the universally acknowledged nomenclature (12).

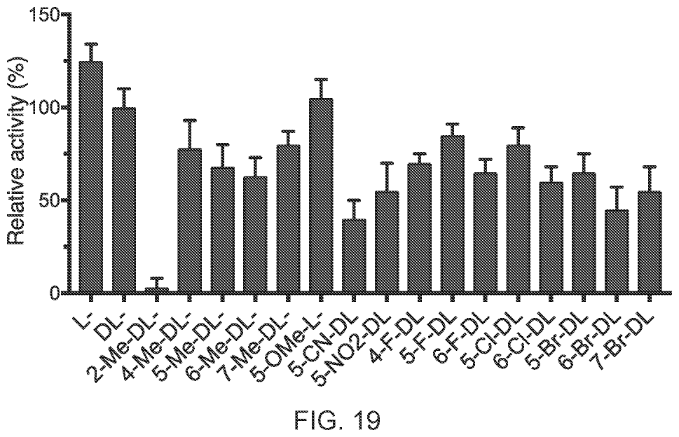

The verified stereoinversion activity of IvoA prompted us to explore its biocatalytic potential. D-tryptophan and its substituted analogues are important building blocks for many peptide pharmaceuticals, such as FDA approved lanreotide, pasireotide, octreotide, macimorelin, triptorelin, etc. Recently, there is growing interest in developing biocatalytic processes for syntheses of substituted D-tryptophans by stereoinversion and deracemization from the L-enantiomers and rac-tryptophans, respectively (21). However, to overcome the entropically unfavorable deracemization process (ΔG⁰=0.4 kcal/mol) (22), the current methods are based on multi-step cascade reactions to establish non-equilibrium conditions for enrichment of D-enantiomers (21). In contrast, IvoA offers a concise one-step, direct nonredox stereoinversion/deracemization process, and allows us to access a library of D-tryptophan analogues in high enantiomeric excess (ee>99%) at millimolar level. Different substitution groups, either electron-withdrawing or electron-donating, at most positions (e.g. positions 4, 5, 6 and 7) on the indole ring can be tolerated (Table 2). No conversion of 2-Me-DL-tryptophan is due to inefficient activation by A domain (FIG. 19), which suggests that substitution at 2-position may interfere with substrate recognition. The poor substrates are generally those with larger substituents (e.g. 5-NO₂, 5-CN, 6-Br, 7-Br), which reflects the size limit by IvoA A domain. In light of recent success in A domain engineering (23), it is conceivable that the substrate scope can be expanded in the future by enlarging the substrate binding pocket of A domain.

In summary, our biochemical study uncovered the unusual activity of IvoA, and our findings expand the function diversity of single-module NRPSs. The reassigned function of IvoA also provides insight to fungal pigment biosynthesis. By inverting the chirality of tryptophan, IvoA perhaps can modulate amino acid flux to pigment biosynthesis in vivo. Considering the proposed role of IvoB and IvoC, one can speculate that the D-configuration generated by IvoA may be retained in the final uncharacterized conidiophore pigment.

TABLE 2
Biocatalytic stereoinversion or deracemization of substituted
tryptophans.^{a}

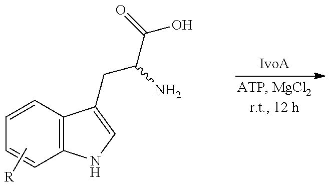

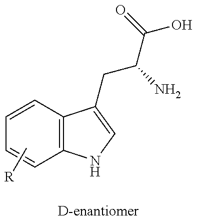

| | Substrate | | |
|---|---|---|---|
| Entry | Stereochemistry | R | ee (%) |
| 1 | L | H | >99 |
| 2 | L | 5-OMe | >99 |
| 3 | rac | 5-CN | 63 |
| 4 | rac | 5-NO₂ | 44 |
| 5 | rac | 4-F | >99 |
| 6 | rac | 5-F | >99 |
| 7 | rac | 6-F | >99 |
| 8 | me | 5-Cl | >99 |
| 9 | rac | 6-Cl | 98 |
| 10 | rac | 5-Br | >99 |
| 11 | rac | 6-Br | 75 |
| 12 | rac | 7-Br | 87 |
| 13 | rac | 2-Me | 1.3 |
| 14 | rac | 4-Me | >99 |
| 15 | rac | 5-Me | >99 |
| 16 | rac | 6-Me | 97 |
| 17 | rac | 7-Me | >99 |

^{a}Expt. Cond.: 1.5 mM substrates, 5 μM IvoA, 5 mM ATP, 10 mM MgCl₂, 100 nM K₂HPO₄ buffer, pH 7.5.

### 1. Materials and Methods

### 1.1. Chemicals and General Methods

L-Tryptophan is purchased from Fisher Chemicals. D-Tryptophan is purchased from Acros Organics. N-acetyl-L-tryptophan and N-acetyl-D-tryptophan are purchased from TCI. N_{α}—Boc-L-tryptophan-N-hydroxy-succinimide ester. N_{α}-Boc-D-tryptophan-N-hydroxy-succinimide ester, and all other tryptophan amino acid derivatives are purchased from Chem-Impex Int'l. Inc. Isopropyl-β-D-1-thio-galactopyranoside (IPTG) was purchased from Carbosynth. Tris-(2-carboxyethyl) phosphine hydrochloride (TCEP-HCl) was purchased from GoldBio Biotechnology. All other chemicals were purchased from Sigma-Aldrich. PCR reactions were performed using the Phusion® high-fidelity DNA polymerase (New England Biolabs) and used according to the manufacturer's instructions. Custom oligonucleotides were synthesized by Integrated DNA Technologies. Escherichia coli strain DH10B was used for cloning procedures.

### 1.2. Protein Expression and Purification

The ivoA gene (AN10576) exon fragments were cloned from the genomic DNA extract of A. nidulans ΔEM strain (1. Liu, N.; Hung, Y.-S.; Gao, S.-S.; Hang, L.; Zou, Y.; Chooi, Y.-H.; Tang, Y. Identification and heterologous production of a benzoyl-primed tricarboxylic acid polyketide intermediate from the zaragozic acid a biosynthetic pathway. Org. Lett. 2017 19, 3560-3563), and assembled through yeast homologous recombination using a Frozen-EZ Yeast Transformation II Kit (Zymo research). Gene fragments were integrated into a 2μ-based yeast expression vector (pXW55) with uracil auxotrophic marker and ADH2 promoter and terminator. To facilitate purification, the target gene was fused with an octahistidine tag at its N-terminus. The full-length wild-type IvoA and mutants were expressed in S. cerevisiae JHY686 strain and expression was autoinduced in YPD medium. Briefly, single colonies of yeast cells harboring plasmids was inoculated into SDCt uracil drop-out culture and left grown at 28° C. for 2 days. The seed culture was then inoculated into YPD culture (1 ml to 50 mL) and left grown at 28° C. for another 2 days. Cells were harvested by centrifugation and washed once with cell lysis buffer (50 mM K₂HPO₄ (pH 7.5), 10 mM imidazole, 300 mM NaCl, 5% glycerol). Cells were flash frozen in liquid nitrogen and lysed by using a stainless-steel Waring blender. The cell lysate was cleared by centrifugation at 26,000 g for 60 min at 4° C. and the supernatant was filtered through a 0.22 μm filter (Millipore). The filtrate was incubated with Ni²⁺-NTA resin for 30 min at 4° C. and then the slurry was loaded onto a gravity column. The resin was washed and eluted with increasing concentrations of imidazole in cell lysis buffer. The fractions were examined by SDS-PAGE gels and targeted proteins were subject to size-exclusion chromatography by using a HiLoad Superdex 200 26/60 column (GE Healthcare) equilibrated in storage buffer (50 mM K₂HPO₄ (pH 7.5), 150 mM NaCl, 1 mM TCEP). Pure fractions were concentrated to 20 mg/mL by Amicon concentrators (Millipore), supplemented with 10% glycerol and stored at −80° C. Protein concentrations were determined by Bradford assay.

For individual domain expression, the expression plasmids were constructed by subcloning the corresponding domain region into a modified pET28a (+) vector (Addgene plasmid #29656). The resulting N-terminal TEV protease cleavable hexahistidine tagged individual domains were overexpressed in E. coli BL21(DE3) cells in LB medium in the presence of 50 mg/L kanamycin. Expression was induced by 100 μM IPTG when OD₆₀₀ reached 1.0 and the cell cultures were left grown at 16° C. overnight. Cells were harvested by centrifugation and lysed by sonication. Purification was performed similarly to the full-length protein.

### 1.3. Fermentation Product Isolation and Purification

The fermentation product was analyzed with a Shimadzu 2020 LC-MS (Phenomenex Kinetex, 1.7 μm, 2.0×100 mm, C18 column) using positive and negative mode electrospray ionization with a linear gradient of 5-95% MeCN—H₂O supplemented with 0.1% (v/v) formic acid in 15 min followed by 95% MeCN for 3 min with a flow rate of 0.3 mL/min. For structural characterization, N-acetyl-D-tryptophan and D-tryptophan were isolated from a 2 L yeast culture overexpressing IvoA protein. The cell pellets containing D-tryptophan were removed by centrifugation and the supernatant containing N-acetyl-D-tryptophan was collected separately.

To purify N-acetyl-D-tryptophan, the pH value of the supernatant was adjusted to 3 by using 1M HCl. The acidified supernatant was extracted with ethyl acetate and the organic layer was combined. The organic solvent was removed by rotavap and the crude extract was dried over Na₂SO₄. N-Acetyl-D-tryptophan was purified by silica-gel chromatography. Fractions containing the target compound were combined and further purified by semipreparative HPLC using a reverse-phase column (Phenomenex Kinetics, C18, 5 μm, 100 Å, 250×4.6 mm). The planar structure of N-acetyl-D-tryptophan was confirmed by comparing NMR spectrum with spectrum reported in the literature and database.^{3 1}H-NMR (500 MHz, CD₃OD): 1.89 (s, 3H), 3.15 (dd, J=14.7, 7.5 Hz, 1H), 3.35 (dd, overlap with solvent, 1H), 4.69 (t, J=14.7, Hz, 1H), 7.00 (ddd, J=8.0, 7.0, 1.0 Hz, 1H), 7.07 (m, 2H), 7.31 (dt, J=8.1, 0.9 Hz, 1H), 7.56 (dt, J=7.9, 1.0 Hz, 1H). The stereochemistry of N-acetyl-D-tryptophan was determined by chiral analytical HPLC with a CHIRALPAK® IA-3 (150×4.6 mm, 3 μm) at room temperature. The mobile phase was 80/20/0.1/9.1 hexanes/ethanol/TFA/DEA and the flow-rate was 1.0 mL/min.

To purify D-tryptophan, the cell pellet was extracted by acetone and the solvent was removed by rotavap. The crude residue was dissolved in mobile phase A (water containing 0.1 (v/v) TFA) and applied to reverse-phase flash-chromatography. Basically, 20 mL of Cosmosil 140 C₁₈—OPN resin (Nacalai Tesque, Inc.) was packed in a Luer-Lock, non-jacketed glass column (Sigma) and equilibrated with mobile phase A. The resin was washed with 3 column volume (CV) of mobile phase and then eluted with increasing methanol content in a step-wise manner. Tryptophan was eluted at 15-25% (v/v) methanol fractions. The pooled fractions were further purified by semipreparative HPLC using a reverse-phase column (Phenomenex Kinetics, C18, 5 μm, 100 Å, 250×4.6 mm). The planar structure of D-tryptophan was confirmed by comparing NMR spectrum with spectrum reported in the literature and database. ¹H-NMR (500 MHz, D₂O): □ 3.37 (dd, J=15.4, 7.8 Hz, 1H), 3.51 (dd, J=15.4, 5.0 Hz, 1H), 4.19 (dd, J=7.7, 5.0 Hz, 1H), 7.20 (ddd, J=8.0, 7.0, 1.1 Hz, 1H), 7.28 (ddd, J=8.2, 7.0, 1.2 Hz, 1H), 7.32 (s, 1H), 7.54 (dt, J=8.2, 1.0 Hz, 1H), 7.72 (dt, J=8.0, 1.0 Hz, 1H). Similarly, L-tryptophan was purified from yeast cells without overexpressing ivoA protein. ¹H-NMR (500 MHz, D₂O): 3.40 (dd, J=15.4, 7.6 Hz, 1H), 3.52 (dd, J=15.4, 5.2 Hz, 1H), 4.26 (dd, J=7.5, 5.0 Hz, 1H), 7.19 (t, J=7.5 Hz, 1H), 7.28 (t, J=7.6 Hz, 1H), 7.33 (s, 1H), 7.53 (d, J=8.1 Hz, 1H), 7.71 (d, J=8.1 Hz, 1H). The stereochemistry was determined by chiral analytical HPLC with a Crownpak® CR(+) column (150 mm×4 mm×3.5 μm, Daicel) at room temperature. The mobile phase was aq. HClO₄ 1% (w/v) supplemented with 15% (v/v) MeOH and the flow rate was 1.0 ml/min.

### 1.4. Enzymatic Assay.

The hydroxylamine-based colorimetric assay for adenylation activity was performed according to the literature (Kadi, N.; Challis, G. L. Chapter 17. Siderophore biosynthesis a substrate specificity assay for nonribosomal peptide synthetase-independent siderophore synthetases involving trapping of acyl-adenylate intermediates with hydroxylamine. Methods Enzymol. 2009, 458, 431-457). Acetyltryptophan acetyltransferase activity was performed by incubating 1-100 μM IvoA with 1 mM D-tryptophan or other substrates with 1 mM acetyl-CoA or 1 mM acetyl-phosphate in 100 mM phosphate buffer (pH 7.5). The reaction mixture was incubated at room temperature and the reaction was quenched at different time interval by mixing with 5-fold volume of methanol. The mixture was clarified by centrifugation to remove protein and salts, and the supernatant was dried in vaccuo by using speedvac. The residue was dissolved in methanol and subjected to LC-MS analysis. For ATP-dependent acetyltransferase activity, 1 mM LD-tryptophan, 5 mM ATP, 1 mM CoA and 5 mM MgCl₂ were used.

The ATP-dependent stereoinversion activity was typically performed with 2-5 μM IvoA, 1 mM L/D-tryptophan, 3 mM ATP and 10 mM MgCl₂ in 100 mM phosphate buffer (pH 7.5), and the reaction was quenched by mixing with 5-volume of methanol. The solvent was removed in vaccuo by speedvac and the residue was dissolved in ethanol and analyzed by chiral-HPLC by using a Crownpak® CR(+) column (150 mm×4 mm×3.5 μm, Daicel) at room temperature. The mobile phase was aq. HClO₄ 1% (w/v) supplemented with 15% (v/v) MeOH and the flow rate was 1.0 ml/min.

When assays were performed in D₂O, enzyme stock solution was buffer exchanged into K₂HPO₄ buffer in D₂O (pD 7.5) by using Zeba™ Spin Desalting Column (ThermoFisher Scientific). All substrates and cofactors were dissolved in the same buffer.

The L-/D-tryptophan loading reactions were performed by incubating 80 μM holo-IvoA-ΔC with 5 mM ATP, 10 mM MgCl₂ and 1 mM L-/D-tryptophan in a final volume of 50 μL. The reaction was allowed to proceed for 15 min before a two-fold dilution with mQH₂O and analysis by UHPLC-ESI-Q-TOF-MS.

The thioesterase activity assay of standalone IvoA-C was performed in ammonium acetate buffer (20 mM, pH=6.9). Typically, 5 mM synthetic substrate (5% DMSO) was incubated with 50 μM enzyme. The reaction was analyzed by HPLC. Boiled enzyme was used as control to measure the background nonenzymatic hydrolysis.

The loaded IvoA-ΔC(E⁰) was prepared enzymatically by incubating holo-enzyme with respective substrate (1 mM) in the presence of excess ATP (5 mM) and MgCl₂ (10 mM) in storage buffer for 2 min. The reaction was quenched by desalting the enzyme through Zeba™ Spin Desalting Column, which is equilibrated in the ammonium acetate buffer (20 mM, pH=6.9). The desalted enzyme was immediately mixed with IvoA-C (50 μM), or boiled enzyme, or chemical hydrolysis (1 M KOH). The hydrolysis reaction was quenched after 1 min by mixing with 2 volume of acetonitrile and subjected to LC-MS analysis.

### 1.5. UHPLC-ESI-O-TOF-MS Analysis of Intact Proteins

The L-/D-tryptophan loading reactions were analyzed on a Bruker MaXis II ESI-Q-TOF-MS connected to a Dionex 3000 RS UHPLC fitted with an ACE C4-300 RP column (100×2.1 mm, 5 μm, 30° C.). The column was eluted with a linear gradient of 5-100% MCCN containing 0.1% formic acid over 30 min. The mass spectrometer was operated in positive ion mode with a scan range of 200-3000 m/z. Source conditions were: end plate offset at −500 V; capillary at −4500 V; nebulizer gas (N₂) at 1.8 bar; dry gas (N₂) at 9.0 L min⁻¹; dry temperature at 200° C. Ion transfer conditions were: ion funnel RF at 400 Vpp; multiple RF at 200 Vpp: quadrupole low mass at 200 m z; collision energy at 8.0 eV; collision RF at 2000 Vpp; transfer time at 110.0 μs; pre-pulse storage time at 10.0 μs.

### 1.6. Genetic Manipulation

The S. cerevisiae hpa3A mutant strain derived from parent JHY686 strain was constructed by integration of a LEU2 marker to the hpa3 loci through homologous recombination. The correct integration was selected by colony-PCR. The resulting strain JHY686-YH (MATα lys2Δ0 his3Δ1 leu2Δ0 ura3Δ0 pep4Δ SAL1⁺ HAP1⁺ CAT5(91M) MIP1(661T) MKT1(30G) RME1 (INS-308A) TAO3 (1493Q) prb1ΔADH2p-npgA-ACSlt hpa3Δ LEU2) was used to transform plasmid overexpressing IvoA protein.

### 1.6 Synthesis of D-Trp-SNAC

N_{α}-Boc-D-tryptophan-N-hydroxy-succinimide ester (0.2 g, 0.5 mmol) was dissolved in anhydrous dichloromethane (10 mL) at room temperature, and to this solution was added N-acetylcysteamine (0.07 g, 0.6 mmol) and diisopropylethylamine (DIPEA, 0.12 g, 1 mmol). This was stirred at room temperature for 2 hrs and washed with saturated ammonium chloride. The organic layer was dried over sodium sulfate and removed by rotavap. The residue was subjected to silica flash chromatography. The resulting white solid product was dissolved in 2 mL of cocktail of 90% trifluoroacetic acid (TFA)/5% water, 5% triisopropylsilane (TIPS) and stirred for 8 hrs. The solvents were evaporated to give a crude oil, which was taken up in minimal volume of dichloromethane and precipitated with diethyl ether. The resulting solid was further washed with diethyl ether to afford the final product in 80% yield. ¹H-NMR (d₆-DMSO, 500 MHz): 11.12 (s, 1H), 8.56 (s, 3H), 8.06 (t, 1H, J=5.3 Hz), 7.55 (d, 1H, J=7.7 Hz), 7.38 (d, 1H, J=8.1 Hz), 7.25 (d, 1H. J=2.5 Hz), 7.10 (ddd, 1H, J=8.2, 7.0, 1.2 Hz), 7.02 (ddd, 1H, J=8.0, 7.0, 1.1 Hz), 4.45 (t, 1H, J=6.6 Hz), 3.27 (m, 2H), 3.15 (q, 2H, J=6.6 Hz), 2.96 (td, 2H, J=6.8, 3.0 Hz), 1.79 (s, 3H). ¹³C-NMR (d₆-DMSO, 125 MHz): □ 196.5, 169.4, 136.3, 127.0, 125.2, 121.3, 118.7, 118.1, 111.7, 106.2, 59.0, 37.8, 28.4, 27.6, 22.6. HRMS ESI m/z calculated for C₁₅H₂₀N₃O₂S⁺ (M+H)⁺ 306.1271, found 306.1258.

### 1.7 Synthesis of L-Trp-SNAC

The synthesis of L-Trp-SNAC is essentially the same as D-Trp-SNAC, except N:-Boc-L-tryptophan-N-hydroxy-succinimide ester was used. ¹H-NMR (d₆-DMSO, 500 MHz): □ 11.14 (s, 1H), 8.61 (s, 3H), 8.06 (t, 1H, J=6.2 Hz), 7.55 (d, 1H, J=8.0 Hz), 7.38 (d, 1H, J=8.1 Hz), 7.25 (s, 1H), 7.10 (t, 1H, J=7.5 Hz), 7.02 (t, 1H, J=7.5, 7.0, 1.1 Hz), 4.44 (t, 1H, J=4.7 Hz), 3.28 (m, 2H), 3.14 (m, 2H), 2.96 (td, 6.7, 2.7, 2H), 1.80 (s, 3H). ¹³C-NMR (d₆-DMSO, 125 MHz): 196.5, 169.5, 136.3, 127.0, 125.2, 121.3, 118.7, 118.1, 111.7, 106.2, 59.0, 37.8, 28.4, 27.6, 22.6. HRMS ESI m/z calculated for C₅H₂₀N₃O₂S⁺ (M+H)⁺ 306.1271, found 306.1264.

### 1.8 Synthesis of D-TrD-Pant

N_{α}-Boc-D-tryptophan-N-hydroxy-succinimide ester (0.1 g, 0.25 mmol) was dissolved in anhydrous dichloromethane (5 mL) at room temperature, and to this solution was added dimethyl ketal protected pantetheine prepared (80 mg, 0.25 mmol)³ and DIPEA, 0.06 g, 0.5 mmol). This was stirred at room temperature for 2 hrs and washed with saturated ammonium chloride. The organic layer was dried over sodium sulfate and removed by rotavap. The residue was subjected to silica flash chromatography. The resulting white-yellow solid was dissolved in 5 mL of cocktail of 75% trifluoroacetic acid (TFA)/20% water/5% triisopropylsilane (TIPS) and stirred for 24 hrs. The solvents were evaporated to give a crude oil, which was taken up in minimal volume of dichloromethane and precipitated with diethyl ether. The resulting solid was further washed with diethyl ether to afford the final product in total 60% yield. ¹H-NMR (d₆-DMSO, 500 MHz): 11.11 (s, 1H), 8.53 (s, 3H), 8.10 (t, 1H, J=5.7 Hz), 7.72 (t, 1H, J=6.1 Hz), 7.55 (d, 1H, J=7.9 Hz), 7.38 (d, 1H, J=8.1 Hz), 7.25 (d, 1H, J=2.4 Hz), 7.10 (t, 1H, J=7.5 Hz), 7.02 (t, 1H, J=7.4 Hz), 4.45 (t, 1H, J=6.7 Hz), 3.70 (s, 1H), 3.31 (m, overlap, 1H), 3.30 (m, overlap, 1H), 3.29 (m, 2H), 3.26 (m, 2H), 3.22 (m, overlap, 1H), 3.18 (m, overlap, 1H), 3.16 (m, 2H), 2.96 (m, 2H), 2.26 (t, 1H, J=8.6 Hz), 0.80 (s, 3H), 0.78 (s, 3H). ¹³C-NMR (dt-DMSO, 125 MHz): 196.5, 172.9, 170.7, 136.3, 126.9, 125.2, 121.3, 118.7, 118.1, 111.6, 106.1, 75.0, 68.0, 59.0, 39.1, 37.7, 35.2, f35.1, 34.8, 28.3, 21.0, 20.3 HRMS ESI m/z calculated for C₂₂H₃₃N₄O₅S⁺ (M+H)⁺ 465.2166, found 465.2193.

Experimental procedures, chromatograms, and spectroscopic data can be found in U.S. Provisional Patent Application Ser. No. 62/902,527 filed on Sep. 18, 2019 and Hai et al. J. Am. Chem. Soc. 2019, 141, 41, 16222, the contents of which are incorporated by reference.

The invention disclosed herein has a number of embodiments. Embodiments of the invention include methods of making a D-tryptophan or a substituted D-tryptophan analog. These methods typically comprise combining L-tryptophan or a substituted L-tryptophan analog with a single-module nonribosomal peptide synthase ivoA polypeptide such that the IvoA polypeptide catalyzes: unidirectional stereoconversion of the L-tryptophan to a D-tryptophan; and/or unidirectional stereoconversion of the substituted L-tryptophan analog to a substituted D-tryptophan analog; so that the D-tryptophan or the substituted D-tryptophan analog is made.

In certain embodiments of the invention, the single-module nonribosomal peptide synthase ivoA polypeptide comprises an amino acid sequence having at least a 90% identity to SEQ ID NO:1. As used herein, “Single-module nonribosomal peptide synthetase IvoA polypeptide” refers to both genetically engineered and naturally occurring enzymes including A. nidulans IvoA polypeptide and enzymes that are related to A. nidulans IvoA polypeptide in sequence but containing amino acid differences. D-tryptophan, for example, can be produced from naturally occurring enzymes that are similar to A. nidulans IvoA polypeptide (see, e.g. SEQ ID NO: 1 or SEQ ID NO: 2). It is known in the art that mutants can be created by standard molecular biology techniques to produce, for example, mutants of SEQ ID NO: 1 that improve catalytic efficiencies or the like. Typically such mutants will have a 50%-99% sequence similarity to SEQ ID NO: 1. In this context, the term “IvoA homologous enzyme” includes a IvoA polypeptide having at least 80%, 85%, 90%, 95%, 97%, 98% or 99% sequence identity with the amino acid sequence set out in SEQ ID NO: 1, wherein the polypeptide has the ability to convert L-tryptophan to D-tryptophan. Such mutants are readily made and then identified in assays which observe the production of a desired compound such as D-tryptophan (typically using A. nidulans IvoA polypeptide (e.g. SEQ ID NO: 1) as a control). These mutants can be used by the methods of this invention to make D-tryptophan or substituted D-tryptophans, for example. Such variants include, for instance, IvoA polypeptides wherein one or more amino acid residues in SEQ ID NO:1 are substituted, added, or deleted.

In some embodiments of the invention, the methodology makes a D-tryptophan. In other embodiments of the invention, the methodology makes a substituted D-tryptophan analog. In certain embodiments of the invention, the substituted D-tryptophan analog comprises a 5-OMe-L-tryptophan, a 4-F-L-tryptophan, a 5-F-L-tryptophan, a 6-F-L-tryptophan, a 5-CI-L-tryptophan, a 6-Cl-L-tryptophan, a 5-Br-L-tryptophan, a 4-Me-L-tryptophan, a 5-Me-L-tryptophan, a 6-Me-L-tryptophan, or a 7-Me-L-tryptophan. In illustrative methods of the invention, the method produces the D-tryptophan or D-tryptophan analog in significant enantiomeric excess, for example where at least 60%-90% of the L-tryptophan or substituted L-tryptophan analog combined in the method is converted to D-tryptophan or a substituted D-tryptophan analog.

In embodiments of the invention, IvoA polypeptides of the invention can be expressed in a heterologous host, for example a heterologous bacteria, yeast or mammalian cell. Polynucleotides encoding such IvoA polypeptides for use in such embodiments can be those known to be present in Aspergillus nidulans (See, e.g. Aspergillus nidulans NT_107011.1 and AN 4641.2) and/or may be modified or synthesized polynucleotides, for example codon optimized polynucleotides useful in a heterologous host (see, e.g. U.S. Patent Publication Nos. 20080154027 and 20110124074 which are incorporated herein by reference). In illustrative methodological embodiments of the invention that are disclosed herein, the D-tryptophan or the substituted D-tryptophan is made via fermentation in a yeast strain selected to overexpress IvoA polypeptide. Optionally, the yeast strain comprises an Aspergillus nidulans phosphopantetheinyl transferase gene; comprises a mutated histone acetyltransferase hpa3 gene; and/or comprises a heterologous leu2 gene. Typically, the yeast strain used in the method produces at least 1 mg/L, 5 mg/L or 10 mg/L of D-tryptophan or substituted D-tryptophan analog.

Embodiments of the invention also include compositions of matter. For example, one embodiment of the invention is a composition of matter comprising a single-module nonribosomal peptide synthase ivoA polypeptide comprising an amino acid sequence having at least a 90% identity to SEQ ID NO:1; and L-tryptophan and D-tryptophan (e.g. where an amount of L-tryptophan in the composition has been converted to D-tryptophan by the single-module nonribosomal peptide synthase ivoA polypeptide); or a L-tryptophan analog and a substituted D-tryptophan analog (e.g. where an amount of the substituted L-tryptophan analog in the composition has been converted to the corresponding substituted D-tryptophan analog by the single-module nonribosomal peptide synthase ivoA polypeptide). In some embodiments, the composition comprises L-tryptophan and D-tryptophan. In other embodiments, the composition comprises a substituted D-tryptophan analog. Optionally, for example, the composition comprises a substituted D-tryptophan having an electron-withdrawing group or an electron donating group at position 4, 5, 6 or 7 on the tryptophan indole ring moiety. In certain embodiments of the invention, the substituted D-tryptophan analog is selected from the group consisting of a 5-OMe-L-tryptophan, a 4-F-L-tryptophan, a 5-F-L-tryptophan, a 6-F-L-tryptophan, a 5-CI-L-tryptophan, a 6-Cl-L-tryptophan, a 5-Br-L-tryptophan, a 4-Me-L-tryptophan, a 5-Me-L-tryptophan, a 6-Me-L-tryptophan, or a 7-Me-L-tryptophan.

In some embodiments of the invention, the composition comprises a yeast such as Saccharomyces Cerevisiae or the like that comprises an exogenous nucleic acid encoding the single-module nonribosomal peptide synthase ivoA polypeptide comprising an amino acid sequence having at least a 90% identity to SEQ ID NO: 1. Optionally, the composition comprises Saccharomyces cerevisiae selected to comprise a mutated histone acetyltransferase hpa3 gene; and/or comprise a heterologous leu2 gene. In certain embodiments of the invention, the composition is a liquid (e.g. a yeast culture medium) and the D-tryptophan or the substituted D-tryptophan analog is present in the composition in amounts of at least 1, 5 or 10 mg/L. Typically, the composition is disposed in a vessel.

Embodiments of the invention further include systems or kits for generating a D-tryptophan or a substituted D-tryptophan analog. Typically these systems or kits comprise a first container comprising a single-module nonribosomal peptide synthase ivoA polypeptide or a polynucleotide encoding a single-module nonribosomal peptide synthase ivoA polypeptide; and a second container comprising a buffer and/or a solution comprising an ATP. In certain embodiments of the invention, the system or kit comprises a yeast strain that overexpresses a heterologous IvoA polypeptide having at least a 90% identity to SEQ ID NO: 1.

### NRPS IvoA Sequences

1. Aspergillus nidulans 1704 amino acid wild type. ACCESSION C8V7P4, Galagan et al., Sequencing of Aspergillus nidulans and comparative analysis with A. fimigatus and A. oryzae. Nature 438 (7071), 1105-1115 (2005).

(SEQ ID NO: 1)
MASPIIQPAGAGIHDIFTQLELWESIDKGLSMITILRDNDVLWKPFLQLTL
FNQLNIVRKAWSATIQKASESDKVPTLKDVYTSESSFIAQALLDTKNLQIT
PPATPRTALSGALLAKTIVIFHHSERAQEELGTELPEEVRSLVNQNAICLK
VLYNANQWHIDLRYKRDSLSSAQAGEVAEIFEQYLEEALEAVASAIPPSPP
VEDDNAGHGGLCKERTDCPKVNRCIHDLIEEQAIARPDQEGICAYDGSLSY
AGLSKLSSVLAEQLKTFGARPEQRVAILMNKSFWYPVVVLAVLKSGAAFVP
LDPSHPKNRLKQLISEIEPCALITTSVLSELADDLGCPSLAIDSDLTRSKE
GSTTALLPNTSASPNNAAYIIFTSGSTGKPKGVVVEHSALSTSAITRGVVL
GLGPDSRVLQYAPHTFDVSVDEILTTLIHGGCVCVPSEDDRFSIAHFMESA
RVTVALLTPTSARTLHPDEVPSLRILQTGGEVLTEDVNDKWSNRVTLFNVY
GPTEASVACVISNRTGLKGAGWVLGQAVGGKLWIVDPDDIERHLPDNEVGE
LVISGAILARGYFRDPSRTESSFVRMRNGERVYRTGDLASMDSAGTIIYHG
RKDLEVKIRGQRINIAEIEIAILQCDLVHSVVVEYPRSGLFEKKLVAVLRF
EDSSSDAEDGLFGGAKGLTEDIYCLLLSHVSSVLTPAMIPSKWLSLPCVPQ
MPSGKADRKQVRGWLEDMDKRTYTRIFHPNGTDNLISDPSDSMVAIWLKVL
KLEPQSLRLDQSFIRNGGDSIMAMEARHQAHEAGINIDVRELLGSRALQEI
GEMATKTSAVEEVSKIEDDRDEPFPLSPVQQMYFDKVSDPSLGLQQRVCVE
IMTKIQPDMLREALNHVIQKHRMLAARFTKHMGQWMQQVPFGKNLKHLSRC
HIYSQAVGSLGDFCSEPMALEDGTLLHAHLQSSGERQTLVLCVHHLVVDFV
SWRVILQDLHDALAAAQNGLPSGISRSTLTFQQWCREQTKYASTLIPEAVL
PFAPGPVNLRFWQPSNVQAVSNTYSEIVQHDFRLSSTQTTQMLEKFTTATV
HPTDLMLATFALAFKRIFTERDTPTIFIEGHGREPWHASLDVSQTVGWFTA
AFPIHLPKDTLLNTTTAILGASERRRSVLANGHPYWACRYLSPNGQKVFGD
DPRHQEMEFVFNYAGSIVQRAPGQTLFAENVRIAEIGHPNCERFSLFDIGA
AIEMPSSELVVSFTFPKGIAHRERVAELVKTYQELLETAVERDLDLSAKLS
SPLVCPADVVRSLEVNGVCIERDVEIVYTPSSIQQHMLWRQSQEPWFYRVQ
GDWTIEKTTTQSEPVDIDRLSHAWNQVVHRHTTLRTVFRYSSEEERFVAIV
LHEVKPAISIIRKGIQTSGSLCRDDDLSPPHRMVLREKDNGSVVCELEFSH
TIIDAASRSIVVQDLLDAYDGKLAHRPLDFPPFWEYIRLAQSSTPSARKEE
LHRAGRVVTLPFQPTHVLSKVPEACKKNEITISSFFMTAWSIVTAKHFVAK
NQRVDSTSSQAVAFDYVLSDRSANIPGIESAVGPYIRLPTLETHVKEGVSL
KNIARGLHAQCTFQSLSQSTQDGSSLELPSKATALQKYSTLVNIRNSGSDS
LDLVSDSGEWKWILQGFSDPWDYDLVFAVNVHAGKVTGWTVEYADGVVEHS
AADEIAKDLNDVVERMVCEII

2. Aspergillus nidulans Variant Having a Histidine Tag.

(SEQ ID NO: 2)
MASHHHHHHHHTASPIIQPAGAGIHDIFTQLELWESIDKGLSMITILRDND
VLWKPFLQLTLFNQLNIVRKAWSATIQKASESDKVPTLKDVYTSESSFIAQ
ALLDTKNLQITPPATPRTALSGALLAKTIVIFHHSERAQEELGTELPEEVR
SLVNQNAICLKVLYNANQWHIDLHYKRDSLSSAQAGEVAEIFEQYLEEALE
AVASAIPPSPPVEDDNAGHGGLCKERTDCPKVNRCIHDLIEEQAIARPDQE
GICAYDGSLSYAGLSKLSSVLAEQLKTFGARPEQRVAILMNKSFWYPVVVL
AVLKSGAAFVPLDPSHPKNRLKQLISEIEPCALITTSVLSELADDLGCPSL
AIDSDLTRSKEGSTTALLPNTSASPNNAAYIIFTSGSTGKPKGVVVEHSAL
STSAITRGVVLGLGPDSRVLQYAPHTFDVSVDEILTTLIHGGCVCVPSEDD
RFSIAHFMESARVTVALLTPTSARTLHPDEVPSLRILQTGGEVLTEDVNDK
WSNRVTLFNVYGPTEASVACVISNRTGLKGAGHVLGQAVGGKLWIVDPDDI
ERHLPDNEVGELVISGAILARGYFRDPSRTESSFVRMRNGERVYRTGDLAS
MDSAGTIIYHGRKDLEVKIRGQRINIAEIEIAILQCDLVHSVVVEYPRSGL
FEKKLVAVLRFEDSSSDAKDGLFGGAKGLTEDIYCLLLSHVSSVLTPAMIP
SKWLSLPCVPQMPSGKADRKQVRGWLEDMDKRTYTRIFHPNGTDNLISDPS
DSMVAIWLKVLKLEPQSLRLDQSFIRNGGDSIMAMEARHQAHEAGINIDVR
ELLGSRALQEIGEMATKTSAVEEVSKIEDDRDEPFPLSPVQQMYFDKVSDP
SLGLQQRVCVEIMTKIQPDMLREALNHVIQKHRMLAARFTKHMGQWMQQVP
FGKNLKHLSRCHIYSQAVGSLGDFCSEPMALEDGTLLHAHLQSSGERQTLV
LCVHHLVVDFVSWRVILQDLHDALAAAQNGLPSGISRSTLTFQQWCREQTK
YASTLIPEAVLPFAPGPVNLRFWQPSNVQAVSNTYSEIVQHDFRLSSTQTT
QMLEKFTTATVHPTDLMLATFALAFKRIFTERDTPTIFIEGHGREPWKASL
DVSQTVGWFTAAFPIHLPKDTLLNTTTAILGASERRRSVLANGHPYWACRY
LSPNGQKVFGDDPRKQEMEFVFNYAGSIVQRAPGQTLFAENVRIAEIGHPN
CERFSLFDIGAAIEMPSSELVVSFTFPKGIAHRERVAELVKTYQELLETAV
ERDLDLSAKLSSPLVCPADVVRSLEVNGVCIERDVEIVYTPSSIQQHMLWR
QSQEPWFYRVQGDWTIEKTTTQSEPVDIDRLSHAWNQVVHRHTTLRTVFRY
SSEEERFVAIVLHEVKPAISIIRKGIQTSGSLCRDDDLSPPHRMVLREKDN
GSVVCELEFSHTIIDAASRSIVVQDLLDAYDGKLAHRPLDFPPFWEYIRLA
QSSTPSARKEELHRAGRVVTLPFQPTKVLSKVPEACKKNEITISSFFMTAW
SIVLAKHFVAHNQRVDSTSSQAVAFDYVLSDRSANIPGIESAVGPYIRLPT
LETHVKEGVSLKNIARGLHAQCTFQSLSQSTQDGSSLELPSKATALQKYST
LVNIRNSGSDSLDLVSDSGEWKWILQGFSDPWDYDLVFAVNVHAGKVTGWT
VEYADGWEHSAADEIAKDLNDVVERMVCEII*

### REFERENCES

(1) (a) Fischbach, M. A. and Walsh, C. T. Assembly-line enzymology for polyketide and nonribosomal peptide antibiotics: Logic, machinery, and mechanisms. Chem. Rev. 2006, 106, 3468-3496. (b) Sieber, S. A. and Mrahiel, M. A. Molecular mechanisms underlying nonribosomal peptide synthesis: approaches to new antibiotics. Chem. Rev. 2005, 105, 715-738.
(2) (a) Ehmann, D. E.; Gehring, A. M.: Walsh, C. T. Lysine biosynthesis in Saccharomyces cerevisiae: mechanism of α-aminoadipate reductase (Lys2) involves posttranslational phosphopantetheinylation by Lys2. Biochemistry 1999, 38, 6176-6177. (b) Hai, Y.: Huang, A. M.: Tang, Y. Structure-guided function discovery of an NRPS-like glycine betaine reductase for choline biosynthesis in fungi. Proc. Natl. Acad. Sci. USA 2012, 109, 21402-21407. (c) Richardt, A.; Kemme, T.; Wagner, S.; Schwarzer. D.; Marahiel, M. A.: Hovemann, B. T. Ebonyl, a novel nonribosomal peptide synthetase for □-alanine conjugation with biogenic amines in Drosophila. J. Biol. Chem. 2003, 278, 41160-41166. (d) Wang, M.: Beissner, M.; Zhao, H. Aryl-aldehyde formation in fungal polyketides: Discovery and characterization of a distinct biosynthetic mechanism. Chem. Biol. 2014, 21, 257-263.
(3) (a) Winkler, M. Carboxylic acid reductase enzymes (CARs). Curr. Opin. Chem. Biol. 2018, 43, 23-29. (b) Ramsden, J. I.; Heath, R. S.; Derrington, S. R.; Montgomery, S. L.; Mangas-Sanchez, J.; Mulholland, K. R.; Turner. N. J. Biocatalytic N-alkylation of amines using either primary alcohols or carboxylic acids via reductive aminase cascade. J. Am. Chem. Soc. 2019, 141, 1201-1206.
(4) (a) Gao, X.: Chooi, Y.-H.; Ames, B. D.; Wang, P.: Walsh, C. T.; Tang, Y. Fungal indole alkaloid biosynthesis: genetic and biochemical investigation of the tryptoquialanine pathway in Penicillium aethiopicum. J. Am. Chem. Soc. 2011, 133, 2729-2741. (b) Shinohara, Y.; Takahashi, S.: Osada, H.; Koyama. Y. Identification of a novel sesquiterpene biosynthetic machinery involved in astellolide biosynthesis. Sci. Rep. 2016, 6, 32865.
(5) (a) Balibar, C. J.; Howard-Jones, A. R.; Walsh, C. T. Terrequinone A biosynthesis through L-tryptophan oxidation, dimerization and bisprenylation. Nat. Chem. Biol. 2007, 3, 584-592. (b) Schneider, P.; Weber. M.; Rosenberger, K.: Hoffmeister, D. A one-pot chemoenzymatic synthesis for the universal precursor of antidiabetes and antiviral bisindolylquinones. Chem. Biol. 2007, 14, 635-644.
(6) Hihner, E.; Öqvist, K.; Li, S.-M. Design of α-keto carboxylic acid dimers by domain recombination of nonribosomal peptide synthetase (NRPS)-like enzymes. Org. Lett. 2019, 21, 498-502.
(7) Yu, X.; Liu, F.; Zou. Y.; Tang, M.-C.: Hang, L.; Houk, K. N.; Tang, Y. Biosynthesis of strained piperazine alkaloids: uncovering the concise pathway of herquline A. J. Am. Chem. Soc. 2016, 138, 13529-13532.
(8) Baccile J. A.; Spraker, J. E.; Le, H. H.: Brandenburger, E.; Gomez, C.: Bok, J. W.; Macheleidt, J.; Brakhage, A. A.: Hoffmeister, D.; Keller, N. P.; Schroeder, F. C. Plant-like biosynthesis of isoquinoline alkaloids in Aspergillus fumigatus. Nat. Chem. Biol. 2016, 12, 419-424.
(9) Sung, C. T.: Chang, S.-L.; Entwistle, R.: Ahn, G.: Lin, T.-S.; Petrova. V.; Yeh, H.-H.; Praseuth, M. B.; Chiang, Y. M.; Oakley, B. R.; Wang, C. C. C. Overexpression of a three-gene conidial pigment biosynthetic pathway in Aspergillus nidulans reveals the first NRPS known to acetylate tryptophan. Fungal Genet. Biol. 2017, 12, 419-424.
(10) Clutterbuck, A. J. A mutational analysis of conidial development in Aspergillus nidulans. Genetics, 1969, 63, 317-327.
(11) McCorkindale, N. J.; Haves, D.: Johnston. G. A.; Clutterbuck, A. J. N-Acetyl-6-hydroxytryptophan a natural substrate of a monophenol oxidase from Aspergillus nidulans. Phytochemistry, 1983, 22, 1026-1028.
(12) Bloudoff, K. and Schmeing, T. M. Structural and functional aspects of the nonribosomal peptide synthetase condensation domain superfamily: discovery, dissection and diversity. Biochem. Biophys. Acta, 2017, 1865, 1587-1604.
(13) Harvey, C. J. B.; Tang. M.; Schlecht, U.: Horecka, J.; Fischer, C. R.: Lin, H. C.: Naughton, B.; Cherry, J.: Miranda, M.; Li, Y. F.: Chu, A. M.; Hennessy, J. R.: Vandova, G. A.; Inglis, D.; Aiyar, R. S.; Steinmetz, L. M.: Davis, R. W.; Medema, M. H.; Sattely, E.; Khosla, C.; St Onge, R. P., Tang, Y.; Hillenmeyer, M. E. Hex: A heterologous expression platform for the discovery of fungal natural products. Sci. Adv. 2018, 11, eaar5459.
(14) Stachelhaus, T.; Walsh, C. T Mutational analysis of the epimerization domain in the initiation module PheATE of gramicidin S synthetase. Biochemistry 2000, 39, 5775-5787.
(15) Bennett, B. D.; Kimball, E. H.; Gao, M.; Osterhout, R.: Van Dien, S. J.; Rabinowitz, J. D. Absolute metabolite concentrations and implied enzyme active site occupancy in Escherichia coli. Nat. Chem. Biol. 2009, 5, 593-599
(16) (a) Yow, G. Y.: Uo, T.; Yoshimura, T.; Esaki, N. D-amino acid N-acetyltransferase of Saccharomyces cerevisiae: a close hoologue of histone acetyltransferase Hpa2p acting exclusively on free D-amino acids. Arch. Microbiol. 2004, 182, 396-403. (b) Yow, G. Y.; Uo, T.; Yoshimura, T.: Esaki, N. Physiological role of D-amino acid N-acetyltransferase of Saccharomyces cerevisiae: detoxification of D-amino acids. Arch. Microbiol. 2006, 185, 39-46. (c) Sampath, V.; Liu, B.; Tafrov, S.; Srinivasan, M.; Rieger, R.; Chen, E. I.; Stemglanz. R. Biochemical characterization of Hpa2 and Hpa3, two small closely related acetyltransferases from Saccharomyces cerevisiae. J. Biol. Chem. 2013, 288, 21506-21513.
(17) (a) Tanner, M. E. Understanding Nature's strategies for enzyme-catalyzed racemization and epimerization. Acc. Chem. Res. 2002, 35, 237-246. (b) Fischer, C.; Ahn, Y-C.; Vederas, J. C. Catalytic mechanism and properties of pyridoxal 5′-phosphate independent racemases: how enzymes alter mismatched acidity and basicity. Nat. Prod. Rep. 2019, in press, DOI: 10.1039/c9np00017h.
(18) Calculated under 0.25 M ionic strength at pH 7 according to Alberty, R. A. Calculating apparent equilibrium constants of enzyme-catalyzed reactions at pH 7. Biochem. Educ. 2000, 28, 12-17.
(19) (a) Hai, Y. and Tang, Y. Biosynthesis of long-chain N-acyl amide by a truncated polyketide synthase-nonribosomal peptide synthetase hybrid megasynthase in fungi. J. Am. Chem. Soc. 2018, 140, 1271-1274. (b) Gao, X.: Haynes, S. W.; Ames, B. D.: Wang, P.: Vien, L. P.: Walsh, C. T.; Tang, Y. Cyclization of fungal nonribosomal peptides by a terminal condensation-like domain. Nat. Chem. Biol. 2012, 8, 823-830.
(20) Müller, S.: Rachid, S.: Hoffmann, T.: Surup, F.: Volz, C.; Zaburannyi, N.: Müller, R. Biosynthesis of crocacin involves an unusual hydrolytic release domain showing similarity to condensation domains. Chem. Biol. Catal. 2014, 21, 855-865.
(21) (a) Schnepel, C.: Kemker, I.; Sewald, N. One-pot synthesis of D-halotryptophans by dynamic stereoinversion using a specific L-amino acid oxidase. ACS Catal. 2019, 9, 1149-1158. (b) Parmeggiani, F.; Casanajo, A. R.; Walton, C. J. W.; Galman, J. L.; Turner, N. J.; Chica, R. A. One-pot biocatalytic synthesis of substituted D-tryptophans from indoles enabled by an engineerred aminotransferase. ACS Catal. 2019, 9, 3482-3486. (c) Matsuyama, A.: Mitsuhashi, K.; Tokuyama, S.; Yamamoto. H. D-aminoacylase and gene encoding the same. U.S. Pat. No. 6,887,697 B2, 2005.
(22) Gruber, C. C.; Lavandera, I.; Faber, K.; Kroutil W. From a racemate to a single enantiomer: deracemization by stereoinversion. Adv. Synth. Catal. 2006, 348, 1789-1805.
(23) (a) Ishikawa, F.; Miyanaga, A.: Kitayama, H.; Nakamura. S.: Nakanishi, I. Kudo, F.: Eguchi, T.; Tanabe, G. An engineered aryl acid adenylation domain with an enlarged substrate binding pocket. Angew. Chem. Int. Ed. Engl. 2019, 58, 6906-6910. (b) Niquille, D. L.; Hansen, D. A.; Mori, T.; Fercher, D.: Kries, H.: Hilvert, D. Nonribosomal biosynthesis of backbone-modified peptides. Nat. Chem. 2018, 10, 282-287.

All publications mentioned herein (e.g. Hai et al. J. Am. Chem. Soc. 2019, 141, 41, 16222; Sung et al., Fungal Genet Biol. 2017 April; 101: 1-6: Von Dohren 2008 Fungal Genetics and Biology 46 Suppl 1(Suppl 1):S45-52; Galagan J E: et al. (2005). “Sequencing of Aspergillus nidulans and comparative analysis with A. fumigatus and A. oryzae”. Nature. 438 (7071): 1105-15; and the references numerically listed above) are incorporated herein by reference to disclose and describe aspects, methods and/or materials in connection with the cited publications.

1. A method of making a D-tryptophan or a substituted D-tryptophan analog comprising:
combining L-tryptophan or a substituted L-tryptophan analog with a single-module nonribosomal peptide synthase ivoA polypeptide such that the IvoA polypeptide catalyzes:
unidirectional stereoconversion of the L-tryptophan to a D-tryptophan;
or unidirectional stereoconversion of the substituted L-tryptophan analog to a substituted D-tryptophan analog;
so that the D-tryptophan or the substituted D-tryptophan analog is made.

2. The method of claim 1, wherein at least 90% of the L-tryptophan or substituted L-tryptophan analog combined in the method is converted to D-tryptophan or a substituted D-tryptophan analog.

3. The method of claim 1, wherein the method makes a substituted D-tryptophan analog.

4. The method of claim 3, wherein the substituted D-tryptophan analog comprises a 5-OMe-L-tryptophan, a 4-F-L-tryptophan, a 5-F-L-tryptophan, a 6-F-L-tryptophan, a 5-Cl-L-tryptophan, a 6-Cl-L-tryptophan, a 5-Br-L-tryptophan, a 4-Me-L-tryptophan, a 5-Me-L-tryptophan, a 6-Me-L-tryptophan, or a 7-Me-L-tryptophan.

5. The method of claim 1, wherein the single-module nonribosomal peptide synthase ivoA polypeptide comprises an amino acid sequence having at least a 90% identity to SEQ ID NO:1.

6. The method of claim 5, wherein the D-tryptophan or the substituted D-tryptophan is made via fermentation in a yeast strain selected to overexpress IvoA polypeptide.

7. The method of claim 6, further wherein the yeast strain:
comprises an Aspergillus nidulans phosphopantetheinyl transferase gene;
comprises a mutated histone acetyltransferase hpa3 gene; and/or
comprises a heterologous leu2 gene.

8. The method of claim 7, wherein the yeast strain produces at least 1 mg/L of D-tryptophan or substituted D-tryptophan analog.

9. A system for generating a D-tryptophan or a substituted D-tryptophan analog comprising:
a first container comprising a single-module nonribosomal peptide synthase ivoA polypeptide or a polynucleotide encoding a single-module nonribosomal peptide synthase ivoA polypeptide; and
a second container comprising a buffer and/or a solution comprising an ATP.

10. The system of claim 9, wherein the system comprises a yeast strain that overexpresses a heterologous IvoA polypeptide having at least a 90% identity to SEQ ID NO: 1.

11. A composition of matter comprising:
a single-module nonribosomal peptide synthase ivoA polypeptide comprising an amino acid sequence having at least a 90% identity to SEQ ID NO:1;
L-tryptophan and D-tryptophan; or
a substituted L-tryptophan analog and a substituted D-tryptophan analog.

12. The composition of claim 11, further comprising Saccharomyces Cerevisiae comprising an exogenous nucleic acid encoding the single-module nonribosomal peptide synthase ivoA polypeptide comprising an amino acid sequence having at least a 90% identity to SEQ ID NO:1.

13. The composition of claim 11, further comprising Saccharomyces Cerevisiae selected to:
comprise a mutated histone acetyltransferase hpa3 gene; and/or
comprise a heterologous leu2 gene.

14. The composition of claim 11, wherein the composition comprises L-tryptophan and D-tryptophan.

15. The composition of claim 11, wherein the composition comprises a substituted D-tryptophan analog.

16. The composition of claim 15, wherein the composition comprises a substituted D-tryptophan having an electron-withdrawing group or an electron donating group at position 4, 5, 6 or 7 on the tryptophan indole ring moiety.

17. The composition of claim 16, wherein the substituted D-tryptophan analog is selected from the group consisting of a 5-OMe-L-tryptophan, a 4-F-L-tryptophan, a 5-F-L-tryptophan, a 6-F-L-tryptophan, a 5-Cl-L-tryptophan, a 6-Cl-L-tryptophan, a 5-Br-L-tryptophan, a 4-Me-L-tryptophan, a 5-Me-L-tryptophan, a 6-Me-L-tryptophan, or a 7-Me-L-tryptophan.

18. The composition of claim 11, wherein the composition is a liquid and the D-tryptophan or the substituted D-tryptophan analog is present in the composition in amounts of at least 1 mg/L.

19. The composition of claim 18, wherein liquid is a yeast culture medium.

20. The composition of claim 11, wherein the composition is disposed in a vessel.
